(19) United States
(12) Patent Application Publication
(10) Pub. No.: US 2005/0214738 A1
(43) Pub. Date: Sep. 29, 2005

(54) Conditional allele system

(76) Inventors: Kryn Stankunas, Menlo Park, CA (US); Jason E. Gestwicki, Mountain View, CA (US); Kelvin Neu, Brooklyn, NY (US); Gerald R. Crabtree, Woodside, CA (US); Joseph Henri Bayle, Redwood City, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/992,304

(22) Filed: Nov. 17, 2004

Related U.S. Application Data
(60) Provisional application No. 60/523,705, filed on Nov. 19, 2003.

(51) Int. Cl. C12Q 1/00; C07H 21/04; C12P 21/04; C12N 15/09; C07K 14/47

(52) U.S. Cl. 435/4; 435/69.7; 435/320.1; 435/325; 530/350; 536/23.5

(57) ABSTRACT

A conditional allelic system is provided where recombinant cells express a fusion protein, where the fusion protein comprises at least a functional portion of a target protein and a destabilizing peptide. The destabilizing peptide binds to a small stabilizing molecule that results in the stabilization of the fusion protein in a functional form, while in the absence of the small stabilizing molecule, the protein function is substantially reduced. The system finds use in studying cellular pathways, preparing transgenic animals that can develop in the presence of the small stabilizing molecule and can then be studied in the presence and absence of the fusion protein to determine the functions and/or effects of target protein function in and/or on various environments. Specifically, a mutated peptide from mTOR is employed as the destabilizing peptide and a modified rapamycin is employed as the small stabilizing molecule.

### CROSS-REFERENCE TO RELATED APPLICATIONS

This application claims priority from provisional application Ser. No. 60/523,705, filed Nov. 19, 2003, which is incorporated herein as if set forth in its entirety.

### STATEMENT REGARDING FEDERALLY SPONSORED RESEARCH OR DEVELOPMENT

This invention was made in part with government support under Grant No. CA39612 awarded by the National Institutes of Health. The government has certain rights in this invention.

### BACKGROUND OF THE INVENTION

1. Field of the Invention

The invention relates generally to protein regulation.

2. Background Information

Biological studies of protein function have largely depended upon manipulation of activity using genetic methods. These include isolation of genetic mutants, targeted deletion of specific genes, or introduction of additional gene copies producing misexpressed, overexpressed, hypermorphic, or neomorphic proteins. Usually, permanent alterations to a genome provide a researcher with no ability to control when the gene(s) the researcher is interested in are active or not. Even existing conditional systems, using, for example, promoter-specific inducible expression or deletion, provide inadequate temporal control. This is because there is a lag between gene expression and protein expression when a gene is induced and a lag between gene deletion and the dissipation of protein activity. These time lags frequently take much longer than the rate of physiological or developmental changes in the biological system being studied. As such, genetic methods do not provide sufficient ability to resolve specific and direct protein functions. This is the goal of modern biology: to understand the dynamic interactions between every protein in every cell type at every time during their development and function. As such, it is imperative to develop new technologies to keep pace with the biological questions being asked. Along with this ability goes the evaluation of compounds as to their effect(s) on cellular function and which of the many cellular proteins and pathways are involved.

Small molecule inhibitors, by acting directly on proteins, provide rapid inactivation and therefore fine temporal control of gene function. In addition, halting drug treatment or the addition of competitor molecules can reverse pharmacological inhibition. Reversible regulation allows for the study of gene function during specific time windows, akin to temperature-shift experiments performed on temperature-sensitive alleles in poikilothermic organisms. For example, the small molecules FK506 and cyclosporin A have been used to define the execution point for calcineurin during mammalian vasculogenesis (Graef, I. A. et al. “Signals Transduced by Ca2+/calcineurin and NFATc3/c4 Pattern the Developing Vasculature.” (2001) Published in Cell 105, 863-875). Treating mice with rapamycin helped reveal a role for FKBP12-rapamycin-associated protein (FRAP), also known as mTor or RAFT, functioning during telencephalon development (Hentges, K. E. et al. “FRAP/mTOR is required for Proliferation and Patterning During Embryonic Development in the Mouse.” (2001) Published in Proc. Natl. Acad. Sci. USA 98, 13796-13801). Unfortunately, chemical genetic studies in animals remain limited by the lack of specific inhibitors for most targets and by the poor pharmacologic profiles of many existing small molecules.

An ideal conditional allele system would combine the high specificity and versatility of genetics with the rapid and reversible nature of small molecule-mediated inhibition. Several methods have been developed that combine chemistry with genetics to provide drug-dependent regulation of tagged or mutated proteins. One innovative method involves the mutation of kinases such that they retain their normal activity but become sensitive to an otherwise benign inhibitor (Bishop, A. C. et al. “A Chemical Switch for Inhibitor-Sensitive Alleles of Any Protein Kinase.” (2000) Published in Nature 407, 395-401). Recently, this concept was used to produce transgenic mice expressing a chemically-sensitive CaMKII in the brain (Wang, H. et al. “Inducible Protein Knockout Reveals Temporal Requirement of CaMKII Reactivation for Memory Consolidation in the Brain.” (2003) Published Proc. Natl. Acad. Sci. USA 100, 4287-4292). In another approach, proteins are expressed as fusions to steroid-binding domains, which causes them to be encapsulated by HSP-90 until they are released by the addition of an appropriate hormone (reviewed in Picard, D. “Posttranslational Regulation of Proteins by Fusions to Steroid-Binding Domains.” (2000) Published in Methods Enzymol. 327, 385-401). In a third method, N-end rule proteolysis of proteins fused to a modified dihydrofolate reductase can be controlled using methotrexate (Johnston, J. A. et al. “Methotrexate Inhibits Proteolysis of Dihydrofolate Reductase by the N-end Rule Pathway.” (1995) Published in J. Biol. Chem. 270(14), 8172-8178; Levy, F. et al. “Analysis of a Conditional Degradation Signal in Yeast and Mammalian Cells.” (1999) Published in Eur. J. Biochem. 259, 244-252). None of these approaches has been applied to an endogenous gene locus. Additional methods used to conditionally remove gene function include targeted protein degradation (Zhou, P. et al. “Harnessing the Ubiquitination Machinery to Target the Degradation of Specific Cellular Proteins.” (2000) Published in Molecular Cell 6, 751-756), ribozymes, antisense RNA, and the emerging technology of RNA interference (RNAi) (reviewed in McManus, M. T. et al. “Gene Silencing in Mammals by Small Interfering RNAs.” (2002) Published in Nat. Rev. Genet. 3(10), 737-747). However, new technologies with faster kinetics and broader applicability are needed.

Based on the principle that induced proximity regulates many intracellular processes, methods have been developed of regulating the activity of proteins by inducing their association using small molecules (Spencer, D. M. et al. “Controlling Signal Transduction with Synthetic Ligands.” (1993) Published in Science 262, 1019-1024). Among other applications, chemical-induced dimerization has been used to regulate cell membrane receptors, non-receptor tyrosine kinases, death inducers, exchange factors, GTPases, and transcription factors. (See Stankunas, K. et al. “Conditional Protein Alleles Using Knockin Mice and a Chemical Inducer of Dimerization.” (2003) Published in Mol. Cell 12, 1615-1624.) To date, however, no method exists that allows dimerization dependent loss-of-function studies. One favored approach is a system where dimerization would regulate protein stability or degradation. Such a method would have the potential to be broadly applied and would allow the creation of fast-acting, small molecule-sensitive loss of function alleles. Desirably, the method should be rapidly responsive to the administration of the small molecule, the molecule should be mildly toxic, if at all, to the cell or host, at a concentration that is effective for the regulation, and the compound should not interfere with other pathways or metabolic processes, so as to obscure the effect of the regulation of the protein target. (The above citations are set forth in Stankunas, K. et al. “Conditional Protein Alleles Using Knockin Mice and a Chemical Inducer of Dimerization.” (2003) Published in Mol. Cell 12, 1615-1624, which is specifically incorporated herein as if set forth in its entirety.)

### RELEVANT LITERATURE

Belshaw, P. J. et al. “Controlling Protein Association and Subcellular Localization with a Synthetic Ligand that Induces Heterodimerization of Protein.” (1996) Published in Proc. Natl. Acad. Sci. USA 93, 4604-4607.
Belshaw, P. J. et al. “Controlling Programmed Cell Death with a Cyclophilin-Cyclosporin-Based Chemical Inducer of Dimerization.” (1996) Published in Chem. Bio. 3, 731-738.
Biggar, S. R. et al. “Chemically Regulated Transcription Factors Reveal the Persistence of Repressor-Resistant Transcription After Disrupting Activator Function.” (2000) Published in J. Biol. Chem. 275, 25381-25390.
Biggar, S. R. et al. “Cell Signaling Can Direct either Binary or Graded Transcriptional Response.” (2001) Published in Embo J. 20, 3167-3176.
Bishop, A. C. et al. “A Chemical Switch for Inhibitor-Sensitive Alleles of Any Protein Kinase.” (2000) Published in Nature 407, 395-401.
Brown, E. J. et al. “A Mammalian Protein Targeted by G1-Arresting Rapamycin-Receptor Complex.” (1994) Published in Nature 369, 756-758.
Chen, J. et al. “Identification of an 11-kDa FkBP12-Rapamycin-Binding Domain Within the 289-kDa FKBP12-Rapamycin-Associated Protein and Characterization of a Critical Serine Residue.” (1995) Published in Proc. Natl. Acad. Sci. USA 92, 4947-4951.
Choi, J. et al. “Structure of the FKBP12-Rapamycin Complex Interacting with the Binding Domain of Human FRAP.” (1996) Published in Science 273, 239-242.
Harding, M. W. et al. “A Receptor for the Immunosuppressant FK506 is a Cis-Trans Peptidyl-Prolyl Isomerase.” (1989) Published in Nature 341, 758-760.
Hentges, K. E. et al. “FRAP/mTOR is required for Proliferation and Patterning During Embryonic Development in the Mouse.” (2001) Published in Proc. Natl. Acad. Sci. USA 98, 13796-13801.
Ho, S. N. et al. “Dimeric Ligands Define a Role for Transcriptional Activation Domains in Reinitiation.” (1996) Published in Nature 382, 822-826.
Johnston, J. A. et al. “Methotrexate Inhibits Proteolysis of Dihydrofolate Reductase by the N-end Rule Pathway.” (1995) Published in J. Biol. Chem. 270(14), 8172-8178.
Kim, D. E. et al. “Protein Structure Prediction and Analysis Using the Robetta Server.” (2004) Published in Nuc. Acids. Res. 32, W526-W531.
Levy, F. et al. “Analysis of a Conditional Degradation Signal in Yeast and Mammalian Cells.” (1999) Published in Eur. J. Biochem. 259, 244-252.
Liberles, S. D. et al. “Inducible Gene Expression and Protein Translocation Using Nontoxic Ligands Identified by a Mammalian Three-Hybrid Screen.” (1997) Published in Proc. Natl. Acad. Sci. USA 94, 7825-7830.
Magari, S. R et al. “Pharmacologic Control of a Humanized Gene Therapy System Implanted Into Nude Mice.” (1997) Published in J. Clin. Invest. 100, 2865-2872.
Marianayagam, N. J. et al. “Fast Folding of a Four Helical Bundle Protein.” (2002) Published in J. Am. Chem. Soc. 124, 9744-9750.
Rivera, V. M. et al. “A Humanized System for Pharmacologic Control of Gene Expression.” (1996) Published in Nat. Med. 2, 1028-1032.
Sabatini, D. M. et al. “RAFT1: A Mammalian Protein that Binds to FKBP12 in a Rapamycin-Dependent Fashion and is Homologous Yeast TORs.” (1994) Published in Cell 78, 35-43.
Spencer, D. M. et al. “A General Strategy for Producing Conditional Alleles of Src-Like Tyrosine Kinases.” (1995) Published in Proc. Natl. Acad. Sci. USA 92, 9805-9809.
Stankunas, K. et al. “Conditional Protein Alleles Using Knockin Mice and a Chemical Inducer of Dimerization.” (2003) Published in Mol. Cell 12, 1615-1624.
Wang, H. et al. “Inducible Protein Knockout Reveals Temporal Requirement of CaMKII Reactivation for Memory Consolidation in the Brain.” (2003) Published Proc. Natl. Acad. Sci. USA 100, 4287-4292.
U.S. Patent Application No. 2004/0170970

### SUMMARY OF THE INVENTION

Methods and compositions are provided that provide for at least one conditional allele that allows for the externally controlled reversible regulation of a target protein in a cell without requiring a temperature change for change in stability. The methods employ systems comprising a nucleic acid sequence encoding a destabilizing polypeptide, either for fusing to a target gene or fused to the target gene as DNA or RNA, and a small organic molecule that stabilizes the fusion protein. The target protein fused to a destabilizing polypeptide is unable to function as the target protein, while, when stabilized, the fusion protein can function as the target protein. By adding a small stabilizing molecule to cells expressing the fusion protein, which small stabilizing molecule acts by itself or in conjunction with an endogenous protein, the fusion protein is stabilized. The cell may lack expression of the endogenous target protein or express the target protein, depending upon the purpose for which the system is being used.

### BRIEF DESCRIPTION OF THE DRAWINGS

FIG. 1. (A) Chemical synthesis and structure of C16- and C20-Methallyl rapamycin. (B) and (C) C20-MaRap exhibits selective affinity for FRB*. SeAP (secreted alkaline phosphatases) reporter activity levels induced by dimerization of either FRB-VP16 (left side) or FRB*-VP16 (right side) to FKBP3-GAL4DBD, were measured in quadruplicate at the indicated concentrations of rapamycin, C16-MaRap, or C20-MaRap and plotted on a graph using GraphPad Prism (GraphPad Software). Error bars represent 95% confidence intervals. EC50 values were determined by fitting a sigmoidal curve to the data using GraphPad Prism and are presented ±95% confidence intervals.

FIG. 2. FRB* destabilizes fusion proteins. (A) Destabilization is primarily a result of the T2098L mutation present in FRB*. Wild-type MEFs (mouse embryonic fibroblasts) were co-transfected with an equimolar mixture of GSK-3β expressing plasmid and one of GSK-3βFRB (left panel), GSK-3βFRB* (middle panel), or GSK-3βFRB# (right panel) expressing plasmids. The transfections were split into multiple wells and treated with cycloheximide (CHX) for the periods shown, as well as with rapamycin or C20-MaRap for 36 h where indicated. The cells were lysed in RIPA buffer and protein expression detected by Western blotting with an anti-GSK-3β antibody. The transfected proteins contain hemagglutinin (HA) epitope tags. HSP-90 and endogenous GSK-3β are used as loading controls. (B) FRB*-mediated reversible destabilization can be widely applied.

The indicated FRB*-fusion proteins were expressed in wild-type MEFs by transfection of corresponding plasmids. Transfections were split into two plates and either left untreated (ND) or treated with 200 nM C20-MaRap for 36 h. Expression of the FRB* fusion proteins was detected by Western blotting lysates with an anti-FRB antibody. The left panel is HSP-90 while the right panel is FRB*, CnBFRB*, EGFPFRB*, GSK3βFRB*, LuciferaseFRB* and HNF-1BFRB*, respectively from top to bottom. (C) and (D) The T2098L mutation reduces the melting temperature of GST-FRB fusion proteins. Recombinant GST-FRB, GST-FRB*, and GST-FRB# proteins were prepared from transformed E. coli and subjected to circular dichroism spectroscopy. (C) The absorbance spectra of GST-FRB* indicates the presence of beta-sheets and alpha-helices. (D) The change in absorbance at 230 nM normalized to standard conditions over increasing temperature was determined and charted for each of GST, GST-FKBP, GST-FRB(wt), GST-FRB*, and GST-FRB#. (E) An equilibrium model of inducible stabilization.

FIG. 3. Inducible Stabilization of GSK-3βFRB*. (A) The GSK-3β^{FRB*} Knock-In Mouse. A schematic of the strategy employed to target the C-terminus of endogenous murine GSK-3β with a FRB* tag. Exon 10 is the last exon of GSK-3β containing translated mRNA and is shown in blue. The darker blue box represents the coding portion of the exon while UTR indicates the untranslated region. (B) Heterozygous GSK-3β^{FRB*} mice express reduced levels of GSK-3βFRB* relative to protein produced by the wild-type allele. 10 μg of thymus (T) or heart (H) RIPA lysate was run on an SDS-PAGE gel and the expression level of GSK-3β was determined by Western blotting. (C) GSK-3βFRB* is destabilized in fibroblasts. Heterozygous MEFs were treated with cycloheximide (CHX) for the indicated periods. The expression level of GSK-3β and GSK-3βFRB* in 10 μg of protein lysate from each condition was detected by Western blotting using an anti-GSK-3β antibody. The blot is overexposed to allow visualization of GSK-3βFRB* at all time points. HSP-70 protein levels serve as a loading control. (D) GSK-3βFRB* is fully stabilized upon C20-MaRap treatment in fibroblasts. MEFs of the indicated genotypes were left untreated (ND) or were treated for 36 h with 100 nM C20-MaRap (MaRap). Protein lysates were prepared and the expression of GSK-3β proteins determined by Western blotting. β-catenin expression is used as a loading control. (E) GSK-3βFRB* expression is restored to wild-type levels with C20-MaRap treatment. Heterozygous GSK-3β^{FRB*} MEFs were treated with 100 nM C20-MaRap for the indicated periods. GSK-3β expression was detected by Western blot analysis of cell lysates. HSP-90 expression serves as the loading control. (F) GSK-3βFRB* stabilization requires FKBP12 binding. Homozygous GSK-3β^{FRB*} MEFs expressing wild-type FKBP12 or carrying an allele of FKBP12 that does not express detectable FKBP12 (FKBP2^{IM}) were left untreated (ND) or were treated with 100 nM C20-MaRap for 36 hours. GSK-3βFRB*, FKBP12, and HSP-90 (loading control) expression was detected by Western blotting with appropriate antibodies.

FIG. 4. GSK-3βFRB* stabilization is reversed with monomeric FK506 (FK506M). (A) FK506M rapidly reverses C20-MaRap induced stabilization of GSK-3βFRB*. Heterozygous GSK-3β^{FRB*} MEFs were left untreated (ND) or were treated with 100 nM C20-MaRap for 36 h. Subsequently, 300 nM FK506M was added for the indicated periods in the continued presence of C20-MaRap. Lysates were prepared and analyzed for GSK-3β and GSK-3βFRB* expression by Western blotting. HSP-90 serves as a loading control. (B) The relative concentration of C20-MaRap to FK506M produces graded stabilization. Similar as (A), except the MEFs were supplemented with the indicated concentrations of FK506M for 9 h following 100 nM C20-MaRap treatment for 36 hours.

FIG. 5. FRB* targets GSK-3βFRB* for proteasomal degradation. Heterozygous GSK-3β^{FRB*} MEFs were treated with the indicated combinations of small stabilizing molecules. In each case that C20-MaRap was used, the cells were first stabilized for 36 h with C20-MaRap, then lactacystin and/or E-64D was added for 30 minutes prior to the addition of FK506 to reverse stabilization for the final 9 h before harvesting. As before, GSK-3β, GSK-3βFRB*, and HSP-90 levels were detected by Western blotting. HSP-90 expression is a loading control.

FIG. 6. GSK-3βFRB* activity is largely restored by inducible stabilization. (A) GSK-3β or GSK-3βFRB* were immunoprecipitated from MEFs of the indicated genotypes and drug treatments and used in [γ-32P]ATP kinase assays using the GSP substrate peptide from glycogen synthase or, in the first lane, the alanine-mutated negative control GSA peptide. Each assay was performed in triplicate and error bars represent one standard deviation. (B) GSK-3βFRB* has kinase activity when bound to FKBP12. A portion of the IP reactions and post-IP supernatant from an IP-kinase assay were saved for Western blot analysis. The levels of GSK-3β, GSK-3βFRB*, and FKBP12 were detected in each condition by Western blotting using respective antibodies. 2 μM of recombinant FKBP12 was added to each IP to ensure GSK-3βFRB* would remain bound to FKBP12-C20-MaRap.

FIG. 7. GSK-3βFRB* is inducibly stabilized in embryos in vivo. A heterozygous GSK-3β^{FRB*} mouse, impregnated by a heterozygous male and carrying E10.0 embryos was mock-injected or injected with C20-MaRap over a 36 hour period before the embryos were isolated at E11.5. The anterior portion of heterozygous embryos was lysed in RIPA buffer, and the expression of GSK-3β and GSK-3βFRB* determined by Western blotting. BRG1 expression serves as a loading control.

FIG. 8. Spatial location and identity of the predicted stabilizing and destabilizing FRB mutants. (A) Changes in solvent accessibility of amino acids after drug binding. Accessibility was calculated using SwissPDBViewer. (B) The primary sequence and structure of amino acids 2090 through 2106 are shown. The three amino acid positions of primary interest to this study (2095, 2098, and 2101) are highlighted in green. The PLF structure was generated in SwissPDBViewer by simple side chain replacement and is shown for illustrative purposes. (C) The targeted amino acids outside the rapamycin interface are highlighted in yellow in the structure of FRB. Note that many of these changes were located near the turn regions of the four-helix bundle. The desired amino acid substitutions and the appropriate amino acid position are listed to the right.

FIG. 9. Intrinsic folding energy of free FRB domains and GST fusions, in vitro, and fold increase in luciferase activity in cells.

FIG. 10. Determination of folding energy and relative contributions of the positions 2095, 2098, and 2101 to stability. (A) Intrinsic destabilizing energy (ΔΔG) of free FRB proteins correlates with the ΔΔG of GST-FRB fusions. (B) The contribution of each site to the stability of the free FRB domain was determined by comparing the ΔΔG of that position while holding others constant. For example, the contribution of leucine at the central position was determined by comparing the luciferase induction in threonine mutants (KTF, ATF, and PTF) to otherwise equivalent mutants with leucine (KLF, ALF, and PLF). The size of the letter indicates the average contribution of that change to stability. Intrinsic folding energy of FRB mutants in cells. (A) The induction of luciferase activity by rapamycin correlates with the intrinsic folding energy of the FRB mutant used to generate the tag. Values are from experiments performed in COS1 cells. The inset shows the similar relationship observed using data from MEF experiments. (B) The contribution of each site to stability was determined by comparing the relative change in fold luciferase induction in COS1 cells.

FIG. 11. FRB* fusion to Pax6 in mice produces an unstable protein that is stabilized with C20-Methallylrapamycin treatment. (A) Design of a fusion allele of three copies of FRB* at the Pax6 locus. Three tandem copies were inserted in exon 4 of the mouse Pax6 gene following the initiating ATG. A neomycin resistance cassette flanked with loxP sites was inserted in intron 3 such that it did not impair splice site usage. Homologous double crossover recombination in ES cells targets the endogenous Pax6 gene. Cross to a mouse expressing cre recombinase from the β-actin promoter deletes the neomycin cassette. (B) FRB*Pax6 levels are low in vivo. A Western blot detects Pax6 in whole cell extracts of cortical neural cultures derived from Pax6^{WT/WT} or Pax6^{FRB*/FRB*} mice. Anti-Heat Shock Protein 90 (HSP90) probing the same blot demonstrates relative protein loading. (C) Degradation kinetics of Pax6 and FRB*Pax6. A Western blot using α-Pax6 antibodies detects Pax6 in extracts of cortical neural cultures from Pax6^{WT/WT} (left panel and first lane, right panel) or Pa₆^{FRB*/FRB*} (right panel) mice. Total protein synthesis was inhibited with 2 μM cycloheximide for the indicated times. (D) Pax6^{FRB*/FRB*} embryo's eyes and nose fail to develop. e14.5 Pax6^{WT/WT} (left) and Pax6^{FRB*/FRB*} (right) littermates. (E) Stabilization of FRB*Pax6 with C20-Methallylrapamycin. Western blot of Pax6 in extracts of cortical neural cultures from Pax6^{FRB*/FRB*} embryos treated with C20-methallylrapamycin at the indicated concentrations for 36 hours. Wild-type Pax6 from Pax6^{WT/Wt} cultures are included at the right for comparison.

FIG. 12. (A and B) Expression of dorsal-ventral marker proteins in transverse sections of branchial spinal cord of cultured whole embryos cultures untreated or treated with C20-methallylrapamycin (2 μM, 48 hours, q 8 hr) as indicated. (A) Engrailed 1 (En1) expression in cultured whole embyos (e8.5 to e10.5). (B) Lim3 expression in cultured whole embyos (e9.5 to e11.5). (C) Expression of Lim3 in transverse sections of caudal spinal cord (r7, r8) of cultured whole embryos cultures started at e9.5 for 48 hours untreated or treated with C20-methallylrapamycin (2 μM, q 8 hr) as indicated at the top. Pax6 (purple) detected against Lim3 in yellow. (D, E) Whole mount visualization of neurofilament in fasciculated axons of e9.5 embryos of the indicated genotypes untreated (D) or treated with 2 μM C20-MaRap 48 hours (E) commencing at e9.5. The hypoglossal (XII cranial) nerve is indicated.

FIG. 13. Reversal of FRB*Pax6 stabilization by competitive inhibition. (A) Schematic of conditional regulation of FRB* tagged proteins. Recruitment of FKBP stabilizes the integrity of FRB* and prevents protein degradation. Competition for FKBP binding by MaRap with FK506M blocks FKBP recruitment and FRB*Pax is destabilized and degraded. (B) Western blot against Pax6 protein in Pax6^{FRB*/FRB*} e13.5 cortical cultures untreated (left lane) or treated with 200 nM C20-Methallylrapamycin for 36 hour followed by treatment with 500 nM FK506M for the indicated timecourse. (C) Transverse sections of caudal hindbrain from cultured whole Pax6^{FRB*/FRB*} untreated (left), treated for 48 hours with 2 μM C20-Methallylrapamycin (middle) or 24 hours with MaRap and 24 hours with FK506M (5 μM). Hindbrain sections were stained with antibody to Lim3 (yellow) and Pax6 (purple). (D) Graphical illustration of experimental definition of temporal windows of protein function with chemical inducers of dimerization. FRB* tagged proteins, in this case Pax6 are unstable and inactive until treatment with rapalog when the protein is rapidly stabilized and active. Reversal of stabilization by competition with FK506M permits a window of protein function to be analysed during development.

FIG. 14 is a table showing the binding of rapalogs as standardized for rapamycin binding with a number of mutants of FRB. The results were obtained using the SeAP assay described in the Experimental section.

FIG. 15. Orthogonal control of GSK3β sub-cellular localization with specific rapalogs. FKBP-GSK3β-GFP is coexpressed with FRB(TLW)-NES and FRB(KTF)-NLS. GFP is visualized in transiently transfected COS1 cells without stimulation where GSK3β is localized to both cell compartments (A) or with 1 hour stimulation with (B) 10 nM C16-BSrap, (C) 10 nM C20-MaRap or (D) 10 nM rapamycin. Cartoons beneath each figure depict the drug-selective recruitment of either TLW-NES or KTF-NLS to FKBP-GSK3β to direct nuclear import or export.

### DETAILED DESCRIPTION OF THE INVENTION

Methods and compositions are provided for employing conditional alleles that allow for the rapid and reversible regulation of a protein function of a target protein. A system is employed originating with a nucleic acid sequence for fusing with nucleic acid encoding a target protein or as a DNA or RNA molecule fused to nucleic acid encoding a target protein. The expression product of the nucleic acid sequence when fused to that encoding the target protein destabilizes the target protein, usually leading to its degradation. Employed with the nucleic acid sequence in the system is a small organic molecule that binds to the expression product and stabilizes the fusion protein by itself or by recruiting an endogenous protein.

The method employs a fusion protein of at least a functional portion of a target protein and a destabilizing polypeptide in a cell. Binding of a small non-toxic stabilizing molecule to the fusion protein in the cell stabilizes the fusion protein to provide a level of function or activity of the target protein greater than in the absence of the stabilizing small molecule, usually stabilizing against degradation. The stabilizing results directly from the binding of the small molecule or as a result of recruitment of an endogeous or co-expressed protein that forms a tripartite complex with the fusion protein and the small molecule. Desirably, for conditional loss-of-function studies, the cells do not independently express the target protein. The cells express the fusion protein, and allow entry of the small stabilizing molecule where the fusion protein is stabilized by the presence of the small stabilizing molecule. Preventing binding of the small stabilizing molecule, e.g. by an antagonist, reverses the stability of the fusion protein.

The method allows for multiple fusion proteins with different stabilizing small molecules that have little or no cross-reactivity with the different destabilizing peptides. Orthogonal analysis can be performed, where one or the other or both of the fusion proteins may be subject to degradation, one or both may be stabilized, and stabilization can occur at different times in the development of cells, in the development of a vertebrate, e.g. mammalian, host, or in the presence of different stimuli, e.g. candidate therapeutic compounds. Because of the rapidity of the response to a stabilizing or destabilizing small molecule, the system is flexible in providing rapid cellular responses.

In the method, recombinant cells expressing the fusion protein are combined with a small molecule, either being investigated for its ability to bind to the destabilizing portion of the fusion protein and stabilize the protein or known for that capability. At least one change in molecular or cellular phenotype is then determined in the presence of the small molecule as indicating that the small molecule stabilizes the activity of the fusion protein, which provides the protein function. Alternatively, where the small molecule is known to stabilize the fusion protein, one can determine the effect of the presence of the activity of the fusion protein, in some cases acting as a surrogate for the activity of the target protein, as compared to its absence or in increasing the level of functional activity for the target protein, on the cell, tissue, or whole organism being studied. These studies can be performed during changes in the environment, which includes natural developmental or physiological events, introduction or removal of a drug, changes in media composition, inhibition of expression of a specific protein, etc.

The cells that are employed may be prokaryotic or eukaryotic, vertebrate or non-vertebrate, mammalian, e.g. mouse or human, and may be in culture, part of a biofilm, tissue, organ, or part of a viable host, may be fetus, neonate, youth or adult, where adult intends that the host has reached puberty. The cells will usually be mammalian cells, such as cells from a mouse, rat, hamster, human, sheep, bovine, porcine, canine, equine, feline, primate, such as human, chimpanzee, gorilla, etc., but may be non-mammalian eukaryotic cells, e.g. avian, frog, flies, worms, yeast, fungi or plant cells or prokaryotic cells, e.g. archaea, bacteria, etc. For hosts, laboratory animals and humans are of interest, where the laboratory animals include mice, rats, rabbits, chickens, sheep, goats, pigs, dogs, cats, non-human primates, etc.

The cells are characterized by expressing the fusion protein, optionally in the absence of expression of the target protein, when the fusion protein will act as a surrogate for the target protein. Therefore, there will be instances where the host cell does not normally express the target protein or where the host cell does express both the target protein and the fusion protein, where the target protein and the target portion of the fusion protein may be the same or different alleles. Where it is desired that the target protein is not expressed, this may be achieved as a result of a genetic knockin of the destabilizing peptide at the target protein site, effectively replacing the target protein with the fusion protein. Alternatively the native, target protein expression could be removed by knockout of the regulatory region and/or the gene encoding the protein, the transcription of an antisense gene or RNAi, or other means that ensures that the native protein is not expressed or has a substantially reduced level of expression.

The cells may be primary cells, cell lines, embryonic stem cells, stem cells, transformed cells, enucleated egg cells with an exogenous nucleus, etc. For multicellular and especially mammalian organisms, the cells may come from any source including tissues, such as brain, eye, skin, liver, bone, marrow, esophagus, muscle, kidney, lymph node, heart, lung, vessel, embryo, blastocyst, etc., differentiated cells, e.g. hematopoietic cells, e.g. lymphocytes, neutrophils, monocytes, macrophages, eosinophils, reticulocytes, etc.; neuronal stem and differentiated cells, e.g. ganglions, neurons, astrocytes, glia, etc., muscle stem cells, e.g. cardiocytes, myoblasts, etc.; skin cells, e.g. epidermal cells, etc.; hair follicle cells; nurse cells, osteoblasts and osteoclasts; epithelial cells; endothelial cells; etc.

In many situations, the cells may have been genetically modified using knockout, knockin, or transgenic expression of genes other than the fusion protein, such as developmental genes, e.g. homeobox genes, genes associated with diseased states, major histocompatibility complex genes, enzymes, particularly hydrolases and kinases, e.g. caspases, metalloproteinases, proteasome components, MAPK cascade, tyrosine kinases, serine/threonine kinases, phosphatases, acetylases, deacetylases, methylases, demethylases, regulators of the cell cycle, e.g. cyclins and cyclin-dependent kinases, transcription factors, genes associated with apoptosis, genes associated with protein and RNA transport, channels, surface membrane protein receptors and ligands, adhesion proteins, structural proteins, e.g. cytoskeletal components.

The hosts may include the fusion protein as a permanent part of their genome or the fusion protein may be introduced into the host to be expressed transiently. The status of the fusion protein and the target protein may be permanent or transient. Where using inhibitory molecules such as antisense and RNAi to remove the native protein, it will be necessary that the fusion protein lack the RNA region that is complemented by the inhibiting RNA. Alternatively, the codons in the targeted RNA region of the fusion protein may be sufficiently modified to prevent the targeting of the RNA without altering the amino acid sequence of the produced protein. For conditional loss-of-function studies, knockin of the sequence encoding the destabilizing polypeptide will be preferred to ensure minimal changes in expression levels or patterns of the fusion protein compared to the native protein. Where knockin methods are not used, the gene encoding the fusion protein may be introduced transiently or under conditions that allow for integration into the genome to provide for stable expression. Possible methods include DNA microinjection, infection with viruses engineered to express the fusion protein, or transfection of DNA that transiently drives the expression of the fusion protein. For many applications, the regulatory region will provide the same spatial and temporal levels of expression as the native target protein. However, the fusion protein may be also introduced to cells by direct intracellular injection or treatment of cells with the fusion protein or a tagged version designed to be taken up by cells. An additional possibility is to introduce expression of the fusion protein by injection or transfection of RNA encoding the fusion protein. In some instances, for example, conditional gain-of-function studies, there will be no need to remove the native expression of the target protein. In these cases, the fusion protein may be introduced by, but not limited to, the methods discussed above.

Thus with a live host, the fusion protein may be introduced into an embryonic cell or fetal cell, may be introduced as cells that have a reasonable lifetime in the host for the purposes for which the mutated cells are used, may be introduced into cells that do not express the target protein, so as to permit the regulated expression of the target protein in the host. Tropic viruses carrying the fusion protein gene with an appropriate regulatory sequence can be used to transfect cells in a host to provide for expression of the fusion protein on an as desired basis.

The fusion protein will be comprised of at least two parts: at least a functional portion of a protein of interest; and a destabilizing polypeptide whose destabilizing effect can be reversed by the addition of a small stabilizing molecule. The destabilizing polypeptide is active over a broad temperature range, generally over a range of 20° C. or greater, so that it can be used at a temperature of interest, e.g. ambient temperatures, e.g. 15-25° C. and also at physiological temperature, e.g. 35 to 40° C. (in the case of mammalian hosts).

There are many peptides that exist naturally or that can be readily modified that will have a destabilizing effect on a protein to which such peptide is fused. So long as the fusion protein is substantially inactivated, conveniently rapidly degraded in the absence of the small stabilizing molecule, the particular mechanism by which the fusion protein is destabilized is not an aspect of this invention. Whether it is improper folding, ubiquitination and degradation in a proteasome, hydrolysis by a proteinase or other mechanism will suffice, so long as the small stabilizing molecule can inhibit the mechanism. Preferably, the destabilizing peptide results in degradation of the fusion protein.

Naturally occurring peptides or modified or mutated naturally occurring peptides can be employed. Alternatively, peptides may be randomly created and tested for their ability to destabilize target proteins. The small molecule may recruit another molecule for its effect or act independently of an endogenous molecule. Depending upon the nature of the small molecule, molecules that may be recruited include albumin, peptidyl-prolyl isomerases, e.g. FKBPs and cyclophilins; ubiquitously expressed molecular chaperones, e.g. Heat Shock Protein 90 (Hsp90); steroid hormone receptors, e.g. estrogen receptors, glucocorticoid receptors, androgen receptors; retinoic acid binding protein, cytoskeletal proteins, such as tubulin and actin; etc., namely those molecules that are generally encountered or will be encountered in the cells containing the fusion protein, where the recruitment would be to the intracellular portion for receptors. See U.S. Patent application Ser. No. 2002/0147133, which is incorporated herein by reference as if set forth in its entirety as for its relevant portions relating to bifunctional molecules, for an extensive list of compounds that can be used to bind to polypeptides that can be employed as destabilizing polypeptides, either as the wild-type or as mutated or randomly created. The destabilization effect should reduce the activity of the fusion protein by at least 50%, preferably at least about 75%, and more preferably by at least about 100%, e.g. degradation, as compared to the fusion protein bound to the stabilizing small molecule. The stabilized fusion protein will usually have at least about 10% of the activity of the target protein, more usually at least about 25%, particularly at least about 50%, and may have up to and including 100% or more.

To enhance degradative instability, naturally occurring peptides may be modified, for example, by modifying the amino acid sequence, e.g. introducing one or a few hydrophobic amino acids in a hydrophilic region or vice versa. For the most part, the hydrophobic amino acids are alanine, leucine, isoleucine, valine, cysteine, methionine, phenylalanine, and tryptophan. Amino acids of intermediate polarity are glycine, serine, threonine, tyrosine, asparagine and glutamine. Amino acids that are polar are aspartic and glutamic acid, lysine, arginine and histidine. The intermediate and polar amino acids may be replaced with hydrophobic amino acids and vice versa to enhance the instability effect of the peptide. One may use an alanine or leucine walk to determine the effect of modification of a polar amino acid on the stability of the peptide and its ability to bind to the small stabilizing molecule and similarly, use a polar amino acid walk, such as threonine or aspartate and determine its destabilizing effect. Appropriate residues to target may be determined by examination of crystal structures for amino acids expected to be surface exposed in the absence of the small molecule and subsequently buried in its presence. Alternatively, candidate mutations may be chosen based on computational methods that predict changes in folding stability. Also, one may use steric effects to destabilize the polypeptide tag, where a small amino acid is replaced with a large amino acid and vice versa, e.g. replacing glycine with leucine, alanine with phenylalanine, and vice versa. As such, clashes between side chains that produce an unstable fusion protein may be produced that are subsequently resolved upon conformational changes introduced upon binding of the small molecule. To screen or select for destabilizing mutations, one may use various cloning techniques, where nucleotides of the peptide are randomly replaced and the resulting mutated sequences introduced into an expression vector and expressed. By dividing each clone, one to which the small stabilizing molecule is added and one where the small stabilizing molecule is absent, one can determine whether there has been instability and whether such instability has been reversed by the small stabilizing molecule. Stabilization of the fusion protein may produce a cellular phenotype that can be readily observed. Alternatively, activity of the fusion protein may be assayed in a lysate using immunoassays, gel electrophoresis, Western blotting, mass spectrometry, etc. For random mutagenesis, usually a high-throughput screening method will be used, such as fluorescent activated cell sorting (“FACS”) or a genetic selection will be employed, where, for example, stabilization of a fusion protein allows for survival on selected growth media, etc.

Peptides that are known to destabilize other proteins include fragments of mTOR, generally of not more than about 200 amino acids, more usually not more than about 100 amino acids, and at least about 50 amino acids, including amino acids 2000 to 2200, particularly a fragment referred to as FRB (An 89 amino acid domain of FRAP-FKBP12-rapamycin-associated protein—capable of binding rapamycin, including amino acid 2098, usually amino acids 2090 to 2110) and mutants thereof. Sites of interest for modification in the binding region, particularly replacing a polar amino acid, that is one having a polar group, such as hydroxyl, carboxyl, amino, or arginine, with a non-polar amino acid, lacking such a polar group, frequently provide for varying degrees of destabilization. Of particular interest as sites are 2090, 2095, 2098, 2101 and 2104. A number of different mutants can be prepared, including L2054A, R2060A, K2066A, R2076A, Y2088A, K2095P,T,K,A, T2098L,F,H, W2101F, Y2104F, and 12111V, particularly T2098L, K2095P, and W2101F, and combinations thereof, and there being not more than a total of about 10 mutations, usually not more than about 5, frequently not more than 3 mutations in this fragment (Chen, J. et al. “Identification of an 11-kDa FkBP12-Rapamycin-Binding Domain Within the 289-kDa FKBP12-Rapamycin-Associated Protein and Characterization of a Critical Serine Residue.” (1995) Published in Proc. Natl. Acad. Sci. USA 92, 4947-4951; Liberles, S. D. et al. “Inducible Gene Expression and Protein Translocation Using Nontoxic Ligands Identified by a Mammalian Three-Hybrid Screen.” (1997) Published in Proc. Natl. Acad. Sci. USA 94, 7825-7830); glucocorticoid receptor (Kalimi, M. et al. “Chloroquine Stabilizes Hepatic Glucocorticoid Receptors.” (1983) Published in Biochem. Biophys. Res. Commun. 112(2), 488-495; Barnett C. A. et al. “In Vitro Stabilization of the Unoccupied Glucocorticoid Receptor by Adenosine 5′-Diphosphate.” (1983) Published in Endocrinology 112(6), 2059-2068); and DHFR (Johnston, J. A. et al. “Methotrexate Inhibits Proteolysis of Dihydrofolate Reductase by the N-end Rule Pathway.” (1995) Published in J. Biol. Chem. 270(14), 8172-8178; Levy, F. et al. “Analysis of a Conditional Degradation Signal in Yeast and Mammalian Cells.” (1999) Published in Eur. J. Biochem. 259, 244-252). As can be appreciated, different mutations will give different levels of destabilization and have different effects on the binding of the stabilizing small organic molecule. Changes can be made in the region of binding to the small organic molecule or outside of the binding region, KSGNVKDLTQAWDLYYH. (SEQ ID NO:1). In addition, other amino acid substitutions can be used instead of the specific mutations described above. In the case of the alanine walk, alanine may be replaced with any of the other non-polar aliphatic or aromatic amino acids, i.e. G, V, L, I, and F. Similarly, K and R, D and E, S and T, and F, Y, and W may be considered to be interchangeable for the purposes of maintaining or modifying the stability of FRB.

As indicated, peptides can be prepared by random mutations from oligopeptides that bind to a small molecule, where the sequence is related to known stable or unstable peptides and be optimized for destabilization, non-interference with function of the target protein and binding to a stabilizing molecule. Also, one can use rational design/structural modeling to predict amino acid substitutions that would be destabilizing.

In addition, proteins that are unstable unless modified or stable except when modified can be used for destabilization. For example, proteins that are stable when phosphorylated, but unstable when not phosphorylated can provide destabilizing oligopeptides, similarly, glycosylated and unglycosylated, acetylated or non-acetylated, etc.

Frequently, where the destabilizing peptide is a naturally occurring peptide, there will be an interest in mutating the peptide and modifying its complementary ligand, if such ligand exists. It will usually be undesirable that the small stabilizing molecule binds to an endogenous protein in the cellular host from which the destabilizing peptide is derived. It has been shown that this can be achieved using a mutated fragment from mTOR and a modified rapamycin, where the modified rapamycin has a much higher affinity for the mutated peptide than for the naturally occurring peptide, while rapamycin has a different binding profile in binding approximately equally well to both FRB and FRB*.

Rapalogs (variants of rapamycin) are described in a number of references, such as Cruz, M. C. et al. “Rapamycin and Less Immunosuppressive Analogs are Toxic to Candida Albicans and Cryptococcus Neoformans via FKBP12-Dependent Inhibition of TOR.” (2001) Published in Antimicrob. Agents Chemother. 45 (11), 3162-3170; Crowe, A. et al. “Absorption and Intestinal Metabolism of SDZ-RAD and Rapamycin in Rats.” (1999) Published in Drug Metab. Dispos. 27(5), 627-632; Geoerger, B. et al. “Antitumor Activity of the Rapamycin Analog CCI-779 in Human Primitive Neuroectodermal Tumor/Medulloblastoma Models as Single Agent and in Combination Chemotherapy.” (2001) Published in Cancer Res. 61(4), 1527-1532; Graziani, E. I et al. “Novel Sulfur-Containing Rapamycin Analogs Prepared by Precursor-directed Biosynthesis.” (2003) Published in Org. Lett. 5(14), 2385-2388; Levy, G. A. et al. “Pharmacokinetics and Tolerability of 40-0-[2-Hydroxyethyl] Rapamycin in De novo Liver Transplant Recipients.” (2001) Published in Transplantation 71(1), 160-163; and Nashan, B. et al. “Early Clinical Experience with a Novel Rapamycin Derivative.” (2002) Published in Ther. Drug Monit. 24(1), 53-58. See also, US2003/0206891.

The destabilizing peptides will usually be at least about 10 amino acids, more usually at least about 20,

frequently at least about 50 amino acids, and not more than about 300, preferably not more than about 150 amino acids. The destabilizing peptide does not interfere significantly with the function of the target protein when bound to the small stabilizing molecule and when appropriate, the recruited protein. Desirably, the destabilizing peptide will be relatively small and may be from an endogenous protein of the host cell or exogenous to the host cell. Therefore, the destabilizing peptide may be drawn from any species. In culture, it will not matter. However, in vivo, where an exogenous protein may result in an immune response, the host, if required, may be tolerized to the fusion protein, where the fusion protein is expressed as an integrated transgene (or knock-in).

Furthermore, for proteins that are known to be unstable, one need not use the entire destabilized protein, but only that portion that provides for both the destabilization and the binding to the small stabilizing molecule. Assays can be used for defining the fragment that provides both of these functions. Fragments to be tested for inducible destabilization can be created by using restriction endonucleases or polymerase chain reaction methods to clone these fragments into the appropriate expression vector. An increase of the activity of the fusion protein comprising the resected gene in an appropriate host in the presence of the small stabilizing molecule would identify those peptides that provided for both destabilization and small stabilizing molecule binding. Activity of the fusion protein could be detected by observation of a cellular phenotype known or designed to appear in the presence of activity of the fusion protein or by testing lysates of the cells for fusion protein activity.

For stabilization of the peptide and fusion protein, different ligands can be used with the different destabilizing peptides. Depending on whether the fusion protein is to be studied in culture, in tissue or in a live host, there will be different restraints on the small stabilizing molecule. In culture, there will be a lower requirement for non-toxicity and binding affinity, as compared to use in vivo, particularly when the fusion protein is present during development of the fetus. In addition, the small stabilizing molecule should have relatively low cross-reactivity with other proteins present in the cells. Other considerations include solubility of the small molecule, stability of the small molecule in aqueous media, serum, in vivo, PK (pharmacokinetics), PD (pharmacodynamics), etc. The small stabilizing molecule may be inorganic or organic, but for the most part, the small stabilizing molecules will be organic molecules, naturally occurring or synthetic. With cloning and combinatorial chemistry, there is a vast opportunity to develop novel peptides that provide destabilization and novel compounds that bind to such peptides and reverse the destabilization and allow the target protein to function. These small stabilizing molecules have a number of characteristics: they will generally be in the range of 200 to 5000 Dal, more usually in the range of about 200 to 1500 Dal; when used in vivo, they should not have significant toxicity to the host, where the toxicity results in a severely sick animal, that is, the level of toxicity should not interfere with the purpose of the investigation and desirably the compound should be substantially non-toxic or have no observable toxicity; they should have a high affinity for the destabilizing peptide, at least about 10⁶M⁻¹, usually at least about 10⁷M⁻¹, and preferably at least about 10⁸M⁻¹, and desirably a substantially smaller affinity, less than about 10⁶M⁻¹, preferably less than about 10⁵M⁻¹for proteins present in the host cells; they should not interfere with the function of the target protein; desirably, there is an antagonist that allows for the destabilization of the fusion protein; should be somewhat soluble in aqueous solutions, should be relatively stable, decent PK/PD, in effect, having analogous properties to the stabilizing molecule, except for the stabilization.

The antagonist may work by competing for binding to the third molecule (like FK506 does for FKBP12), or by binding the initial small molecule (competing with FRB* for example), or by binding the protein but not affecting instability.

Considering the individual proteins, small stabilizing molecules that can stabilize the mutated FRB derivatives, FRB*(PLF) and FRB#(KLW), include rapamycin and “rapalogs,” that is, derivatives of rapamycin that retain at least a significant portion of the binding affinity of rapamycin, and may have other virtues, such as lower toxicity, ease of crossing the blood-brain barrier, solubility, stability, PK, PD, etc. Derivatives that have found use include alkyl derivatives of from 2 to 8 carbon atoms, usually 4 to 6 carbon atoms at the 16 or 20 position of rapamycin. Other derivatives are described in US2003/0206891, which describes a number of rapalogs, which disclosure is incorporated by reference herein, as if set forth in its entirety. For steroid binding receptors, the naturally occurring steroid or its analogs will find use in activating the steroid receptor, where the steroid receptor is inactivated by the binding of HSP-90 and binding of the steroid to its receptor results in the release of HSP-90 with activation of the receptor. Useful compounds for glucocorticoid receptor include glucocorticoids. Useful compounds for DHFR include methotrexate and derivatives of methotrexate.

In some instances, the small stabilizing molecule may act in concert with another molecule. For example, with the rapamycin compounds, the rapamycin not only binds to FRB, but also binds to FKBP proteins, including FKBP12. Since FKBP12 is substantially ubiquitous in eukaryotic cells and hosts, the need for co-expression of FKBP is not a problem, since FKBP12 is substantially always present. Expression of FKBPs, including FKBP12, can be induced using transfections, infections, making transgenes, etc. With other destabilizing peptides, where the stabilizing effect of the small stabilizing molecule involves an endogenous protein, it will be necessary to ensure that the endogenous protein is present in the cells of interest, particularly where one is interested in developmental proteins. As indicated previously, for recruitment, bifunctional molecules can be prepared that include one component for binding to the destabilizing peptide and one component for recruiting the other protein.

The subject constructs find use in any situation where one is interested in the conditional presence of a protein in a viable cell, particularly where the cells are involved in the development of a host, such as a mammalian host, or in being able to control the amount of a protein in the cells of a host using a small stabilizing molecule. In addition, by varying the amount of the small stabilizing molecule, stabilization and function of the fusion protein can be varied in proportion to the amount of available small stabilizing molecule in the cell. Alternatively, one may use a small stabilizing molecule antagonist, where the addition of the antagonist results in regulating the amount of the fusion protein.

One component of the subject invention is the genetic construct, which in one aspect can be used for homologous recombination. This construct may include an expression construct for selection of transformants, portions of the locus to define the site at which homologous recombination is to occur, and the gene encoding the destabilizing peptide to be in reading frame with the target gene. If selection is involved, various genes can be used for selection, including toxins, e.g. antibiotics, such as G418, neomycin, etc., genes providing for detection, e.g. β-galactosidase, etc. Desirably, the selection genes can be inactivated or removed from the host genome. The latter can be achieved using specific targeted recombination termini and a corresponding recombinase, e.g. LoxP sequences and Cre enzyme. The enzyme can be introduced into the embryonic stem cells or germ cells of a whole animal after selection and identification of the occurrence of homologous recombination.

Other genes may be present in the construct for various reasons, such as manipulations, identification, isolation, etc., of the nucleic acid or the encoded protein. Thus, other peptides may be included as epitopes for which good antibodies exist, for performing immunoassays, polyhistidines, polypeptide biotin mimics, etc.

As indicated previously, only the functional portion of the target protein is required. However, in most cases, the homologously recombining construct will be designed such that the fusion protein produced by the targeted locus will include the intact target protein or a partially truncated protein, usually truncated by less than about 20% of the amino acids present, more usually truncated by not more than about 10%. Conveniently, the destabilizing peptide gene may be inserted at or within about 20,

more usually within 10, codons of the start or stop codon. The construct will be flanked by nucleotide sequences homologous with the nucleotide sequence at the site of insertion. Generally, the flanking sequences will be at least about 20 nt, more usually at least about 1 kb, and may be 5 kb or more, usually not more than about 15 kb, as a matter of convenience. Additional nucleotides, corresponding to the targeted locus may be present separating the selection gene from the destabilizing gene, generally not exceeding 500 nt, usually not exceeding 200 nt. The total size of the construct will generally be at least about 5 kb, more usually at least about 10 kb, and not more than about 50 kb, usually not more than about 20 kb. Techniques for knockin and knock-out constructs are described in: Tsuzuki, T. et al. “Embryonic Stem Cell Gene Targeting Using Bacteriophage Lambda Vectors Generated by Phage-Plasmid Recombination.” (1998) Published in Nucleic Acids Res. 26(4), 988-993; Aoyama, C. et al. “Bacteriophage Gene Targeting Vectors Generated by Transplacement.” (2002) Published in Biotechiques 33 (4), 806-810, 812; Storck, T. et al. “Rapid Construction in Yeast of Complex Targeting Vectors for Gene Manipulation in the Mouse.” (1996) Nucleic Acids Res. 24(22), 4594-4596; Wilson, C. J. et al. “Yeast Artificial Chromosome Targeting Technology: An Approach For the Deletion of Genes in the C57BL/6 Mouse.” (2001) Published in Anal. Biochem. 296(2), 270-278; Yang, Y. et al. “Site-Specific Gene Targeting in Mouse Embryonic Stem Cells With Intact Bacterial Artificial Chromosomes.” (2003) Published in Nat. Biotechnol. 21(4), 447-451; Testa, G. et al. “Engineering the Mouse Genome with Bacterial Artificial Chromosomes to Create Multipurpose Alleles.” (2002) Published in Nat. Biotechnol. 21(4), 443-447.

Earlier techniques have been developed to enhance homologous recombination efficiency. For example, positive and negative selective genes may be employed where homologous recombination results in loss of the positive selection gene. By raising the transformants in an environment that selects for the loss of the positive selection gene, the homologously recombined cells are greatly enriched. One can then select for the negative selection gene, where one selects for those cells that include this gene. See, for example, Thomas, K. R. et al. “Site-Directed Mutagenesis by Gene Targeting in Mouse Embryo-Derived Stem Cells.” (1987) Published in Cell 51(3), 503-512; U.S. Pat. No. 5,574,205; and references cited therein.

For transient expression or production of transgenic animals expressing the fusion protein, other constructs can be provided. In these cases, the construct will include genetic elements to express the target protein, a truncated, but active, target protein, or a mutated target protein fused to the destabilizing, small-molecule binding, polypeptide. The mutations may provide hyperactivity or new activities to the target protein. The construct will also include promoter, enhancer, or other regulatory elements that direct the expression of mRNA corresponding to the fusion protein. The directed expression may mimic the native expression of the target protein or may express the fusion protein ectopically or at abnormal levels. The construct may include intron(s) and/or exon(s), regulatory regions, or portions thereof of the target gene, splice sites, signal leader sequence, transmembrane sequence, or other functional sequences for recombination or processing of the mRNA, translation and translocation. These sequences will depend upon the nature of the target gene and the purpose of the transformation.

The construct free of extraneous nucleic acid or vectors may be used for introduction of the construct into a cell. The vectors include plasmids, attenuated or defective DNA virus, such as but not limited to, herpes simplex virus (HSV), papillomavirus, Epstein Barr virus (EBV), adenovirus, adeno-associated virus (AAV), and the like. Defective viruses, appropriately packaged, which entirely or almost entirely lack viral genes, are preferred. Defective virus is not infective after introduction into a cell. Use of defective viral vectors, particularly tropic for particular cell types, allows for administration to cells of a particular type, even where mixtures of cells exist. Specific viral vectors include: an attenuated adenovirus vector, such as the vector described by Stratford-Perricaudet et al. (“Widespread Long-Term Gene transfer to Mouse Skeletal Muscles and Heart.” (1992) Published in J. Clin. Invest. 90(2), 626-630); and a defective adeno-associated virus vector (Samulski, R. J. et al. “A Recombinant Plasmid from Which an Infectious Adeno-Associated Virus Genome can be Excised in Vitro and its Use to Study Viral Replication.” (1987) Published in J. Virol. 61(10), 3096-3101; Samulski, R. J. et al. “Helper-Free Stocks of Recombinant Adeno-Associated Viruses: Normal Integration Does Not Require Viral Gene Expression.” (1989) Published in J. Virol. 63(9), 3822-3828).

The construct containing DNA or RNA may be introduced in vitro by lipofection. For the past decade, there has been increasing use of liposomes for encapsulation and transfection of nucleic acids in vitro. (Felgner, P. L. et al. “Lipofection: a Highly Efficient Lipid-Mediated DNA-Transfection Procedure.” (1987) Published in Proc. Natl. Acad. Sci. USA 84(21), 7413-7417; see Machy, P. et al. “Gene Transfer from Targeted Liposomes to Specific Lymphoid Cells by Electroporation.” (1988) Published in Proc. Natl. Acad. Sci. USA 85(21), 8027-8031). The use of cationic lipids may promote encapsulation of negatively charged nucleic acids, and also promote fusion with negatively charged cell membranes (Felgner, P. L. et al. “Cationic Liposome-Mediated Transfection.” (1989) Published in Nature 337, 387-388). It is also possible to introduce the vector in vitro as a naked DNA plasmid, using calcium phosphate precipitation or other known agent. Alternatively, the vector containing the genetic construct can be introduced via a DNA vector transporter (see, e.g., Wilson, J. M. et al. “Hepatocyte-Directed Gene Transfer in Vivo Leads to Transient Improvement of Hypercholesterolemia in Low Density Lipoprotein Receptor-Deficient Rabbits.” (1992) Published in J. Biol. Chem. 267(2), 963-967; Wu, G. Y. et al. “Receptor-Mediated Gene Delivery and Expression in Vivo.” (1988) Published in J. Biol. Chem. 263(29), 14621-14624; Hartmut et al., Canadian Patent Application No. 2,012,311, filed Mar. 15, 1990). The genetic construct can be introduced into the desired host cells in vitro by methods known in the art, e.g., transfection, electroporation, microinjection, transduction, cell fusion, DEAE dextran, calcium phosphate precipitation, lipofection (lysosome fusion), use of a gene gun, using a viral vector, with a DNA vector transporter, and the like.

There are numerous ways to construct genetic constructs that apply regardless of the purpose of the construct. For expression constructs and descriptions of other conventional manipulative processes, see, e.g., Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); Glover, D. N., ed. “DNA Cloning: A Practical Approach,” Volumes I and II (1985) IRL Press, Oxford; Gait, M. J. ed.,“Oligonucleotide Synthesis: A Practical Approach” (1984) IRL Press, Oxford; Harnes, B. D. and Higgins, S. J., eds. “Nucleic Acid Hybridization: A Practical Approach” (1985) IRL Press, Oxford; Hames, B. D. and Higgins, S. J., eds., “Transcription And Translation: A Practical Approach” (1984) IRL Press, Oxford; Freshney, R. I. “Animal Cell Culture: A Practical Approach” (1986) IRL Press, Oxford; Woodward, J. “Immobilized Cells And Enzymes: A Practical Approach” (1986) IRL Press, Oxford; Perbal, B., ed., “A Practical Guide To Molecular Cloning” (1984) John Wiley and Sons, New York.

Established cell lines, including transformed cell lines, are suitable as hosts. Normal diploid cells, cell strains derived from in vitro culture of primary tissue, as well as primary explants (including relatively undifferentiated cells such as hematopoetic stem cells) are also suitable. Candidate cells need not be genotypically deficient in a selection gene so long as the selection gene is dominantly acting. The host cells preferably will be embryonic stem cells or established mammalian cell lines.

Cell lines may be modified by knocking out specific genes, introducing specific genes, enhancing or diminishing the expression of a protein or the like. The modification may be transient, as in the case of introduction of antisense DNA or dsRNA or may be permanent, by deleting a gene, introducing a gene encoding the antisense mRNA of the target protein, adding a dominant recessive gene, or the like. Research animals may be employed of various strains, where the strains are a result of naturally occurring mutations and breeding or using genetic modifications of embryonic or other cells with a resulting genetically modified host, which may be vertebrate, e.g. mammalian, fish, insect, or the like, or non-vertebrate, e.g. nematode, etc.

Knockout mice are extensively described in the literature. One may use cells of any of the organs from the intact host for the purposes of this invention. Illustrative of the development of knockout and knockin mice are Nozawa, S. et al. “Characteristics of Immunoglobulin Gene Usage of the Xenoantibody Binding to Gal-Alpha(1,3)gal Target Antigens in the Gal Knockout Mouse.” (2001) Published in Transplantation 72(1), 147-155; Ferreira C. et al. “Ferritin Knockout Mice: A Model of Hyperferritinemia in the Absence of Iron Overload.” (2001) Published in Blood 98(3), 525-532; Kotani, N. et al. “Knockout of Mouse Beta 1,4-Galactosyltransferase-1 gene Results in a Dramatic Shift of Outer Chain Moieties of N-Glycans From Type 2 or Type 1 Chains in Hepatic Membrane and Plasma Glycoproteins.” (2001) Published in Biochem. J. 357(Pt 3), 827-834; Zhou, D. et al. “Effects of NF-kappaB1 (p50) Targeted Gene Disruption on Ionizing Radiation-Induced NF-kappaB Activation and TNFalpha, IL--1alpha, IL--1beta and IL-6 mRNA Expression in Vivo.” (2001) Published in Int. J. Radiat. Biol. 77(7), 763-772; and Chang, H. et al. “Studying TGF-Beta Superfamily Signaling by Knockouts and Knockins.” (2001) Published in Mol. Cell. Endocrinol. 180(1-2), 39-46, and references cited therein, to provide only a few of the large number of publications concerning genetically modified mice.

In addition one may use hybridomas, where a first cell having the desired gene(s) is fused with an immortalized cell under conditions where the chromosomes from the first cell are stably maintained. The gene(s) could be transcription factors, proteins of interest, e.g. human proteins in a non-human host cell, or provide for enhanced expression of a protein.

Target proteins of interest may be proteins present at any stage of development or many stages of development. The target proteins may be secreted or non-secreted. Of particular interest in live hosts are proteins involved with development at the early stages of development, e.g. fetus and neonate, where the proteins are also involved in pathways of cells in a mature host, particularly where the pathways may be associated with disease. Proteins associated with development, particularly the mammalian paralogs, include homeobox proteins including Hox proteins, transcription factors including Engrailed, Pax6, Nkx2 family members, GSK-3α and -3β genes in the synthetic pathway of retinoic acid, chromatin modifying proteins including histone methyltransferases, histone acetylases, histone deacetylases, Polycomb group (PcG) proteins, SWI/SNF and related complex containing proteins, parvin, doppel, components of NFAT signaling (calcineurin, NFAT, Dyrk), components of the Wnt signaling pathway (Wnt, Fz, Axin, Dishevelled, APC, β-catenin, etc.), components of the Notch signaling pathway (Notch, Delta, Serrate, Su(H), etc.), components of hedgehog signaling pathways (Sonic hedgehog and related proteins, patched, Gli, etc.), components of receptor tyrosine kinase signaling pathway (EGF, EGFR, Ras, Raf, Mek ERK, Elk-1 etc.), and components of TGF-beta signaling pathways (TGF-beta, Dpp, BMPs, respective receptors, Smads, etc.). Of the protein categories, transcription factors, inhibitors, regulatory factors, enzymes, membrane proteins, structural proteins, and proteins complexing with any of these proteins, are of interest. Specific proteins include enzymes, such as the hydrolases exemplified by amide cleaving peptidases, such as caspases, thrombin, plasminogen, tissue plasminogen activator, cathepsins, dipeptidyl peptidases, prostate specific antigen, elastase, collagenase, exopeptidases, endopeptidases, aminopeptidase, metalloproteinases, including both the serine/threonine proteases and the tyrosine proteases; hydrolases such as acetylcholinesterase, saccharidases, lipases, acylases, ATP cyclohydrolase, cerebrosidases, ATPase, sphingomyelinases, phosphatases, phosphodiesterases, nucleases, both endo- and exonucleases; oxidoreductases, such as the cytochrome proteins, the dehydrogenases, such as NAD dependent dehydrogenases, xanthine dehyrogenase, dihydroorotate dehydrogenase, aldehyde and alcohol dehydrogenase, aromatase; the reductases, such as aldose reductase, HMG-CoA reductase, trypanothione reductase, etc., and other oxidoreductases, such as peroxidases, such as myeloperoxidase, glutathione peroxidase, etc., oxidases, such as monoamine oxidase, myeloperoxidases, and other enzymes within the class, such as NO synthase, thioredoxin reductase, dopamine β-hydroxylase, superoxide dismutase, nox-1 oxygenase, etc.; and other enzymes of other classes, such as the transaminase, GABA transaminase, the synthases, β-ketoacyl carrier protein synthase, thymidylate synthase, synthatases, such as the amino acid tRNA synthatase, transferases, such as enol-pyruvyl transferase, glycinamide ribonucleotide transformylase, COX-1 and -2, adenosine deaminase. Kinases are of great significance, such as tyrosine kinases, the MAP kinases, the cyclin dependent kinases, GTP kinases, ser/thr kinases, Chk1 and 2, glycogen synthase kinases-3α and 3β, etc.

Also of interest are enzyme inhibitors, such as α1-antitrypsin, antithrombin, cyclophilin inhibitors, proteasome inhibitors, etc.

Other proteins of interest are the oncogenes, such as Src, Ras, Neu, Erb, Fos, Kit, Jun, Myc, Myb, Abl, Bcl, etc, cytokines, such as the interferons, α-γ, interleukins, 1-19, and integrins, adhesins, TNF, receptors, hormones, colony stimulating factors, growth factors, such as epidermal growth factor, fibroblast growth factor, etc., bone morphogenetic proteins, developmental proteins, such as the Hox proteins, or other proteins binding to or regulating proteins binding to homeoboxes, e.g. the hedgehog proteins, basement membrane proteins, heat shock proteins, proteins containing Kruppel and Kringle structures chaperonins, calcium associated proteins, e.g. calmodulin, calcineurin, etc., membrane channels, transporter proteins, etc.

Also of interest are the proteins associated with cell growth, proliferation and cell cycle progression, such as the mTor, TSC2, cyclins, cyclin dependent kinases, p53, RB, etc. Also of interest are neuronal proteins, such as β-amyloid, TNF, prion, APP, transporters, e.g. dopamine transporter, receptors, such as NMDA receptors, AMDA receptors, dopamine receptors, channels, etc.

Another class of proteins is the transcription factors and their inhibitors or regulatory proteins, such as Adr Ace, Amt, AP, Atf, Att, Baf, Brn, Btf, C Ebp, C Jun, C Ets, CREB, CF, Chop, DP, E2F, Elk, Gata, Hnf, Iii A-H, Irf, NY Y, Otf, NFkB, NF-AT, Oct-1, Pea, Pit, PU, S, SP, Stat, Tef, TFIII, TFIIII, Ubf and Usf, while the inhibitors include Id proteins, Erk, IkB, LIF, Smad, RANTES, Tdg, etc., as well as other proteins associated with pathways that induce transcription factor synthesis, activation or inhibition. Another class of proteins that are of interest are the surface membrane proteins, where many of such proteins are receptors, such as G protein complex receptors, hormone receptors, interleukin receptors, steroid receptors, transporters, etc. These receptors include insulin receptor, glucose transporter, IL-2, 4, etc. receptor, chemokine receptors, e.g. CXCR4, PPAR, etc. Also, the MHC proteins can be of interest.

In some instances, housekeeping proteins will be of interest, such as the proteins involved in the tricarboxylic acid cycle, the Krebs cycle, glycogenesis, chaperones, basal transcription factors, DNA replication enzymes, translation machinery components, proteins involved in subcellular targeting, etc.

In the case of secreted proteins where the host may not be readily accessible, e.g. fetus, one may have an interest in being able to modulate the amount of the secreted protein available to the cells or host. In this way, one can see the effect of the availability or lack thereof of the secreted protein on the host at various stages of development. Of course, there will be other situations, even where the cells are accessible, where one would be interested in being able to modulate the activity of a secreted protein in relation to the cellular development.

Various pathways will be of interest associated with the different proteins. Thus, pathways involving signal transduction as a result of ligand binding to a surface membrane protein receptor, vesicle formation and transport, multistage synthesis of cellular components, proteasomes, peroxisomes, spindle formation, tubulin assemblage, processing of ingested compounds, e.g. toxins, drugs, etc.

The cells comprising the subject constructs may be used to identify proteins associated with a pathway of interest, the effect of a change in environment, such as the presence of a drug or drug candidate, on the production of the protein of interest, the effect of the presence, absence or amount of a protein on the phenotype of the cell, changes in the regulation of expression, the regulation by a receptor of a cellular pathway and to that extent, compounds that affect the transduction of a signal by the receptor, the effect of the target protein on host development, the effect of the target protein on cellular physiology (including, e.g. cell cycle progression), the effect of the target protein on initiation or progression of a disease state, etc. The cells may also be used for screening for small molecules that can serve to bind to and stabilize the fusion protein. These small molecules can then be screened for binding to the source of the oligopeptide, where the source is a naturally occurring peptide.

In cells in culture or live hosts, one may maintain the target protein by adding the small stabilizing molecule to the medium or host and rapidly or slowly reverse the presence or amount of the target protein by adding an antagonist of the small stabilizing molecule or withdrawing or terminating the addition of the small stabilizing molecule. For culture, usually one need only add the small stabilizing molecule or antagonist to the culture at the desired concentration and one can remove the medium to rapidly withdraw the small stabilizing molecule or antagonist. For a live host, one may administer the small stabilizing molecule or antagonist in a variety of ways, individual bolus, periodic administration, infusion, injection, slow release pills or capsules, etc., where the administration may be orally or parenteral, including subcutaneous, intramuscularly, intraarterially, intravenously, peritoneally, etc.

Depending on the nature of the target protein, various methods can be used to determine the effect of the target protein on other proteins. One can use immunoassays to determine changes in the amount of a protein of interest with variations in the amount of the target protein. A number of immunoassays are available for use with a lysate, where one can obtain a determination of the level of the target protein and any protein of interest whose amount is related to the level of the target protein. Alternatively, one may use gel or capillary electrophoresis, Western blotting or mass spectrometry to identify changes, where these methods allow for the determination of a plurality of proteins of interest simultaneously. Other techniques are described in the literature presented above, as well as in the patent and scientific literature. The particular method employed will depend upon the nature of the sample, whether there are a few or numerous samples, the equipment available, the sensitivity of the methodology, the nature of the experiment, etc. Where the protein of interest is in the cell membrane, it may be assayed with an intact cell (using flow cytometry or other methods) or lysate. Where the protein of interest is intracellular, then a lysate will usually be used. Where the protein of interest is secreted, it can be determined in the culture medium or in the fluid into which it is secreted in the host.

The cells for assaying may come from a culture, whole animals, dissected tissues, biopsy, scrape, surgery, exsanguination, blood sample, saliva sample, cerebral spinal fluid, cord blood, placenta, etc.

As indicated, the fusion proteins as conditional protein alleles find particular use in mammalian developmental studies, such as development of the blastocyst, embryonic disk, conceptus, neural cord, somite, organs, such as the brain, sex organs, liver, spleen, lungs, heart, bone, muscle, blood, thyroid, thymus, bladders, etc. However, all studies of dynamic physiological events, including cell cycle progression, apoptosis, axonal outgrowth, synapse formation, lymphocyte activation, ulcer formation, sclerotic plaque, inflammation, formation of amyloid plaque and fibrillary tangles, etc, would benefit from applications of this conditional allele system. The subject method can be used in these systems to minimize secondary effects and/or aid in separating primary from secondary effects arising from changes in target protein activity. This feature is especially valuable for genomic or proteomic experiments where indirect effects can obscure the identification of direct targets of the protein whose function is gained or lost.

Destabilizing peptides binding to different small stabilizing molecules are available and different ones can be prepared. By using combinations of fusion proteins one can regulate the times at which the fusion proteins are functional during the developmental period of a mammalian host. By employing stabilizing molecules and antagonists for the stabilizing molecules, the functioning of the fusion proteins can be varied from about zero to about 100% over relatively short times. In this way one can determine the period(s) when the function of the fusion protein is essential, how the two fusion proteins interact at various times and when the function of the fusion protein is no longer necessary. By examining the fetus, one can determine the organs and compartments affected by the under or over production of the target protein as naturally expressed and/or expressed as the fusion protein.

The subject system can be expanded to evaluate a number of different features related to translocation in a cell, transcriptional activation, vesicle inclusion, or other phenomena. By using fluorescent proteins, one can identify the site of the protein in the cell, particularly as to localization in the nucleus, mitochondria, cytoplasm, endoplasmic reticulum, cell membrane, etc. By attaching localization sequences that direct the protein into and out of the nucleus, cytoplasm, cell membrane, endoplasmic reticulum, etc., one can provide for determining the site of the fusion protein or the recruited protein in a particular cellular compartment. One can provide for the presence of one of the two protein components of the subject system in a compartment and observe the effect of transport of the other component to that compartment. Since one can turn the fusion protein on and off, one can follow the effect of the fusion protein in such compartment. In addition, one may modify the recruited protein so that it is fused to a protein of interest that will interact with the fusion protein. By being able to control the level of the fusion protein present in the cell, one can effectively titrate the effect of the interaction of the target protein portion of the fusion protein with the protein of interest.

The subject system can also be applied to gene therapy. By modifying cells, such as hematopoietic stem cells, myoblasts, fibroblasts, neuronal stem cells, etc. and transforming them with a construct according to this invention, one can control the expression of the subject protein and proteins that the subject protein in turn controls. Particularly, by having cells where the target gene has been knocked out or is otherwise non-functional (perhaps naturally and resulting in the treated pathology), by introducing the subject construct one can provide a surrogate gene that can be regulated with a small stabilizing molecule. Also, by having a transcription factor as a conditional allele, transcription of one or more genes can be controlled, so as to produce or prevent the production of a protein. Where the protein is an endocrine, paracrine or autocrine secreted protein, regulation can be greatly extended throughout the host. Thus by taking the small stabilizing molecule orally, one can induce the stabilization of a protein, such as insulin, glucose transporter, angiotensin, ACE, an adhesion protein, caspase, etc., depending upon what disease problem is being alleviated. For examples of gene therapy, see, WO 03/030821 and WO 02/077186.

The convenience of the subject system is very valuable. By using the subject inducible stabilization conditional allele system, when it is desirable to remove gene function for an extended period of time, one need only cease drug delivery. If rapid onset of termination is desired, one need only add an antagonist to the small stabilizing molecule to provide for rapid degradation. In a developing mammal, if one is interested in the long-term adult effects of loss-of-function but needs to bypass a developmental requirement, the stabilizing small stabilizing molecule only needs to be applied during the early developmental window to allow the mammal to survive to adulthood. One can evaluate the effect of the absence of the protein during the development, where the result may be the formation of a teratogen, loss of one or more limbs, aneuploidy of some of the cells, inadequate development of one or more organs, structural changes of the host, etc.

The “drug on” nature of the subject system is useful for drug target validation studies because it mimics the action of a pharmacologic compound. Protein stability can be controlled in the adult in a reversible manner and the level of activity can be titrated to achieve a therapeutic window. Since the subject system is genetically based, it can be readily combined with various disease models to test whether removing the function of the gene will cause or prevent disease phenotypes. Additionally, the effects of removing gene function can be assayed even after the disease is apparent or at different stages of disease severity. The exemplary GSK-3β has been hypothesized to have roles in several disease conditions, including type II diabetes, bipolar disorder and neurodegenerative disease. The subject GSK-3β^{FRB*} allele is valuable for validating the clinical use of GSK-3β modifying therapeutics in mouse models of these and other diseases. The following examples are intended to illustrate but not limit the invention.

### EXPERIMENTAL

### Experimental Procedures

C20-MaRap Synthesis

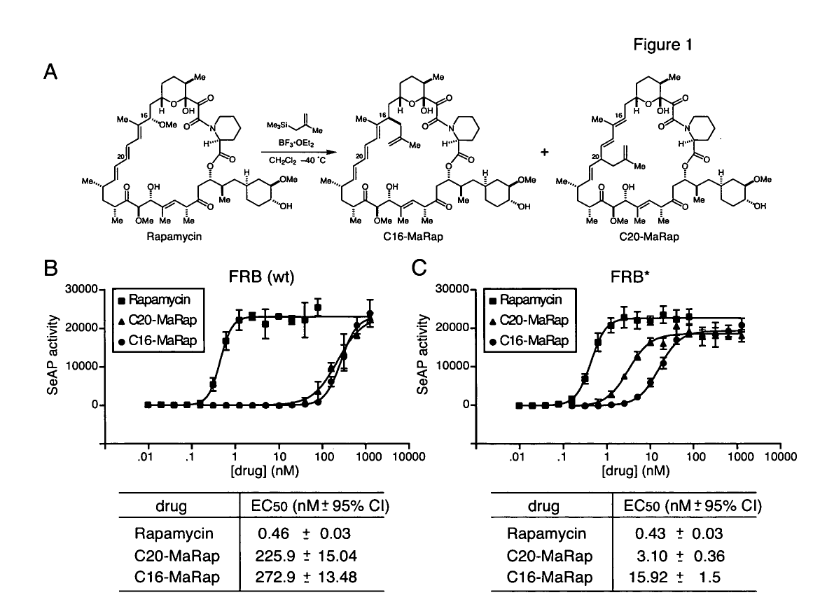

C-20 MaRap synthesis was carried out by dissolving Rapamycin (400 mg) and trimethylmethallylsilane (2.3 mL) in dichloromethane (30 mL) and cooled to −40° C. Boron trifluoride etherate (3.38 mL) was added in one portion, and the reaction was stirred at −40° C. and monitored by analytical HPLC. The reaction was quenched after 5 h with aqueous saturated sodium bicarbonate, warmed to room temperature, and the organic products were extracted into dichloromethane. Silica-gel chromatography (2:1 to 1:1 to 1:3 hexanes: ethyl acetate) gave 201 mg of MaRap as a mixture of three isomers by analytical HPLC: C20-(R or S)-methallylrapamycin (isomer A), C20-(R or S)-methallylrapamycin (isomer B), and C16-(R)-methallylrapamycin (isomer C). The compounds were purified by reverse-phase HPLC using a ternary solvent system. HPLC solvents and the column (Waters Xterra M8 phenyl 5 mm, 19×100 mm column) were preheated in coiled tubing using a 50° C. water bath, with a flow rate of 17 mL/min. The mobile phase consisted of an isocratic mixture of 9 parts of 80% CH3OH/H2O and 1 part of 20% CH3CN/H2O. The desired product, C20-MaRap (63 mg, tr=11.7 min) was isolated along with the other C20-MaRap diastereomer (isomer A, tr=10.5 min) and C16-(R)-MaRap (isomer C, tr=14.5 min). C20-MaRap was characterized using 1H NMR and mass spectrometry. (FIG. 1)

Reporter Assays

Transcription activation by recruitment of chimeric transcriptional activation and DNA binding domains is described. COS-1 cells were electroporated with 2 m each of the following expression plasmids (Ho, S. N., Biggar, S. R., Spencer, D. M., Schreiber, S. L., and Crabtree, G. R. “Dimeric ligands define a role for transcriptional activation domains in reinitiation.” (1996) Nature 382, 822-826): pG5IL2SX (drives transcription of bacterial secreted alkaline phosphatase (SeAP) through GAL4 DNA binding 22 elements), pBJ5-GF3E (which expresses the GAL4 DNA binding domain fused to three copies of human FKBP12), and pBJ5-FRBVP. (which expresses a FRB fusion with the Herpes Simplex Virus VP16 Activation domain) A FRB*-VP16 fusion protein which consists of modified FRB protein fused to the VP16 activation domain was also used selectively. Transfected cells were distributed equally to 96 well plates and were incubated at 37° C. for 24 to 36 hours before media was supplemented with drug in a dilution curve in which each point is represented in quadruplicate. Cells were incubated at 37° C. for an additional 24 hours followed by a 2-hour treatment at 65° C. to inactivate endogenous phosphatases. Each well was supplemented with 100μL of 1 mM methylumbelliferylphosphate dissolved in 2 M diethanolamine buffered to pH 10.0 with carbonate and incubated at 37° C. for 16 hours. Fluorescence was measured with a Fluoroscan (ICN) plate reader at 355 nm transmission and 455 nm emission using Deltasoft software.

Mouse Genetics

A GSK-3β^{FRB*} knock-in mouse was created in the following manner. A selectable plasmid was constructed containing mouse genomic DNA from the 3′ end of the GSK-3β locus, where a loxP-flanked neomycin-resistance cassette was cloned into the last intron, and a FRB*HA sequence was inserted in place of the normal stop codon. (FIG. 3) This plasmid was electroporated into TC1 ES cells and drug resistant lines were screened for correct targeting using Southern blots of restriction digested DNA and radio-labeled probes outside of each targeting arm. Mice were derived from the targeted mice ES cells using standard techniques. The neomycin-resistance cassette was deleted by crossing germline-transmitting mice with mice expressing the Cre recombinase under the β-actin promoter (Lewandoski, M. et al. “Analysis of Fgf8 Gene Function in vertebrate Development.” (1997) Published in Cold Spring Harb. Symp. Quant Biol. 62, 159-168). Deletion of the cassette was confirmed by PCR. The final derived mouse line was screened for the absence of the Cre transgene and maintained on a CD1 outbred background.

Cell Culture

MEFs cell lines were prepared from E14.5-E16.5 embryos of indicated genotypes by eviscerating the embryos in HBSS, treating the remaining tissue with 0.2% collagenase and trypsin, and plating the cells in DMEM containing 10% FCS, antibiotics, and 0.1 mM β-mercaptoethanol. Once established, the MEFs were cultured using standard techniques and treated with various small molecules as indicated.

MEF Transfections

MEFs were transfected using FuGENE-6 (Roche Applied Science). Plasmid DNA (5 μg per 6 cm dish) was mixed in a 1:2 ratio with the FuGENE-6 reagent in Opti-MEM (Invitrogen Life Technologies) before being applied to the cells growing in DMEM/10% FCS and containing C20-MaRap where appropriate. After 12 hours, the cells were split to 6 well plates before further drug treatments to ensure each well contained an equivalently transfected population.

Western Blotting

Western Blot analysis was carried out using cell lysates prepared in RIPA buffer (10 mM Tris pH 7.2, 150 mM NaCl, 0.1% SDS, 1% Triton X-100, 1% deoxycholate, 5 mM EDTA, protease inhibitor cocktail (Invitrogen Life Technologies)) and then separated using standard SDS/PAGE before being transferred to PVDF (Millipore) using standard Western blotting procedures. The anti-GSK-3β antibody, used at 1:2500, the anti-HSP-90 antibody, 1:2500, and the anti-FKBP12 antibody, 1:2500, were purchased from BD Transduction Labs. The anti-β-catenin antibody, 1:2000, and anti-HSP-70 antibody, 1:1000 were purchased from Santa Cruz Biotechnology. The anti-BRG1 antibody, 1:1000, was previously prepared in our laboratory (Khavari, P. A. et al. “BRG1 Contains a Conserved Domain of the SWI2/SNF2 Family Necessary for Normal Mitotic Growth and Transcription.” (1993) Published in Nature 366, 170-174). In all cases, Western blots were incubated overnight with the appropriate primary antibodies in TBST (10 mM Tris 8.0, 150 mM NaCl, 0.1% Tween-20) containing 10% dry milk, and then washed 3 times with TBST. The blots were subsequently incubated for 30 minutes with HRP-conjugated secondary antibodies from Jackson Immunoresearch, washed 3 more times with TBST, and then developed using standard ECL reagents (Amersham Biosciences) and Kodak XAR film.

Circular Dichroism Spectroscopy

Circular Dichroism Spectroscopy was used to determine the stability of the fusion proteins. FRB variants were amplified by PCR and ligated into pGEX2T as in-frame fusions to generate pGEX-FRB(wt), pGEX-FRB*, and pGEX-FRB#. A pGST-FKBP12 vector was previously described. These vectors were used to transform chemically competent Escherichia coli BL21 cells to derive the strains: GST-FKBP, GST-FRB(wt), GST-FRB*, and GST-FRB#. Proteins were induced by the addition of IPTG and purified using glutathione-sepharose beads (Amersham Biosciences) by standard techniques. Pooled washes from the final elution steps were dialyzed against CD buffer (10 mM Tris-HCl pH 7.2, 100 mM NaCl, 1 mM DTT). Protein concentrations were determined by absorbance at 280 nm. Purity was >80% by SDS-PAGE. Dialyzed samples were diluted to 0.25 mg/mL in CD buffer. Freshly prepared samples were used as significant precipitation was observed after prolonged storage at −80 or 4° C. under these buffer conditions. The circular dichroism spectra of diluted samples was monitored on an AVIV 62A DS spectrometer. For Tm determinations, elliptisity was monitored at 230 nm while temperature was varied from 50° C. to 75° C. with 1° C. steps. Equilibration time was 1 minute. Nonlinear regression fits were performed using GraphPad Prism (GraphPad Software). The reported values are the mean of two independent experiments.

Immunoprecipitation Kinase Assays

Immunoprecipitation kinase assays carried out with the MEF cell lines indicated above. Cells were lysed in mild lysis buffer (20 mM Tris 7.2, 150 mM NaCl, 1 mM EDTA, 0.5% NP-40, 5% glycerol, 100 nM C20-MaRap, 100 nM microcystin-LR (Calbiochem), and protease inhibitor cocktail). 400 μg of lysate was buffer-exchanged into immunoprecipitation (IP) buffer (10 mM Tris 7.2, 150 mM NaCl, 1 mM EGTA, 100 nM C20-MaRap, 100 nM microcystin, and protease inhibitor cocktail), supplemented with 2 μM purified recombinant FKBP12, prepared using standard GST-fusion purification methods. IP reactions were incubated at 4° C. for 1.5 hours with 125 ng of anti-GSK-3β, followed by a 2 h incubation with 20 μL of a 1:4 dilution of Protein-G Sepharose Fast Flow (Amersham Biosciences) in Sepharose CL-4B (Amersham Biosciences). GSK-3β activity was assayed in kinase buffer (20 mM Tris 7.2, 10 mM MgCl2, 1 mM DTT, 100 nM C20-MaRap, 1 mM peptide, 0.5 mM ATP) by measuring [γ-32P]ATP (Amersham Biosciences) incorporation into the phospho-glycogensynthase peptide-2 (GSP) or the negative control glycogen synthase peptide-2[Ala 21] (GSA) (Upstate). The reactions were spotted on P81 paper (Whatman), washed with 125 mM phosphoric acid, and measured by liquid scintillation.

Whole Animal Studies

GSK-3β^{+/FRB*} mice were intercrossed and the date of vaginal plug observation was set as E0.5. At E9.5, the stage of embryonic development was confirmed by ultrasonography (Chang, C. P. et al. “Sonographic Staging of the Developmental Status of Mouse Embryos in Utero.” (2003) Published in Genesis 36, 7-11) and mice were weighed. Drug administration commenced at E10.0. C20-MaRap was dissolved as a stock in N,N-dimethylacetamide (DMA) and was diluted into a delivery vehicle containing 10% Polyethylene glycol 400, 17% Polyoxyethylene sorbitan monooleate and 10% DMA to a final concentration of 100 mg/kg. 100 μL was immediately injected intraperitoneally (IP) to pregnant females with injections repeated twice more at 12 hour intervals to E11.5 when mice were sacrificed, and embryos and organs were dissected and frozen in N2(1). Tissues anterior to the embryonic forelimbs of individual embryos were used for preparation of protein extracts with the remainder used to determine the concentration of drug and for genotyping.

Cloning of FRB Mutants

Point mutations in the FRB sequence were introduced by Quikchange mutagenesis using vectors pS-Luc-FRB or pBJ5-FRB-VP16 as templates. Overlapping oligos were designed and used according to manufacturer's specification (www.stratagene.com). Sequences encoding the FRB mutants were amplified by PCR from the pS-Luc-FRB vector and ligated as C-terminal fusions into the BamH1 and EcoR1 sites of pGEX2t (Amersham Pharmacia). Mutant identities were confirmed by sequencing.

Protein Expression and Tryptophan Fluorescence

GST-FRB protein was purified from Escherichia coli BL21 and dialyzed extensively into 10 mM Tris pH 7.2 with 150 mM NaCl. The pGEX2t vector contains one unique thrombin recognition site between the GST and FRB domains, which permits the cleavage of the GST using the biotinylated thrombin kit (Novagen). Protein concentrations were determined by Bradford assay using BSA as a standard. All proteins were adjusted to 1000 nM in the dialysis buffer and 50 μL aliquoted into CoStar black opaque 96-well plates. To initiate denaturation, 50 μL of 2×GuHCl was added, the solution mixed and incubated 15 minutes at room temperature. Tryptophan fluoresence (280 nm/330 nm) was measured in a SpectraMax M2 (Molecular Devices, Sunnyvale, Calif.). Fluorescence values at eleven GuHCl concentrations in triplicate were used to calculate ΔG values in PRISM 3 (GraphPad Software) according to the equations in reference: Marianayagam, N.J. et al. “Fast Folding of a Four Helical Bundle Protein.” (2002) Published in J. Am. Chem. Soc. 124, 9744-9750.

Luciferase Stabilization Assay

COS1 cells were cultured in DMEM+10% FCS and penicillin/streptomycin at 37° C. and 5% CO2. For transfection experiments, DNA was resuspended in 20 volumes of OptiMEM and vortexed briefly prior to addition of 3 volumes of FUGENE 6 (Roche). After a 15 min. incubation, the mixture was applied dropwise to 10 cm plates containing approximately 80% confluent COS1 cells. The following day, cells were divided into the wells of 24-well culture plates in 500 μL volume of culture media. Rapamycin was added after cells regained adherence. Following a 24 hour incubation, media was removed, cells washed once in cold PBS, and luciferase assays performed using the Dual Luciferase Kit (Promega, Madison, Wis.). Experiments were repeated at least 3 times in either duplicate or triplicate wells. The fold induction values were normalized to an internal standard consisting of Luc-PLF-transfected cells treated with 5 nM rapamycin. Similar conditions were used in experiments that utilized MEFs.

Preparation of FRB*Pax6 Mice.

Two pBluescript (Stratagene) constructs containing murine Pax6 genomic sequences were obtained from Dr. Peter Gruss (Max Planck Institute, Gottingen, Germany). One (5.5 kb) included non coding exons 1-3 and the other (˜9 kb) contained all the coding exons (Ventura, A. et al. “Cre-Lox-Regulated Conditional RNA Interference From Transgenes.” (2004) Published in Proc. Natl. Acad. Sci. USA 101(28), 10380-10385; Stankunas, K. et al. “Conditional Protein Alleles Using Knockin Mice and a Chemical Inducer of Dimerization.” (2003) Published in Mol. Cell 12, 1615-1624; Kim, D. H. et al. “Raptor and mTOR: Subunits of a Nutrient-Sensitive Complex.” (2004) Published in Curr. Top Microbiol. Immunol. 279, 259-270). Exon 4 containing the start site of translation was subcloned in Litmus 29 (New England Biolabs) and mutated in vitro at position −3 to create a XhoI site. This mutation was not predicted to influence protein translation. Three tandem copies of FRB* were cloned into this site to create an in frame genomic gene fusion. This cassette was cloned back into the exon 4-7 genomic clone. A portion of intron 3 was cloned into the exon 4-7 genomic clone with the site of discontinuity located 300 bp upstream of exon 4 to prevent an influence on mRNA splice site usage. These two clones were inserted as knock-in arms in the pLNTK vector containing HSV thymidine kinase and the neomycin phosphotransferase genes controlled by the mouse phosphoglycerate kinase promoter. This FRB*Pax6 knock-in contruct was electroporated in TC1 embryonic stem cells and one clone with homologous recombination at the Pax6 locus and containing the FRB* insertion was identified by Southern blot. Mice were derived from chimeric blastocyts injected with these ES cells. F1 from these chimerae had the Small eye phenotype at the expected frequency.

Cortical Culture

e13.5 embryos were harvested from a Pax6^{FRB*/+} intercross and cortical hemispheres isolated from individuals identified by eye and craniofacial morphology and later confirmed by genotype specific PCR. Pia were removed and neocortex dissected from lateral geniculate nuclei in Hank's balanced salt solution (HBSS). Tissue was minced roughly and placed in centrifuge tubes containing HBSS+20% foetal calf serum. Tissue pieces were allowed to settle and the media was replaced first with HBSS+serum then HBSS alone. The tissue was incubated with 0.1% trypsin+2 mM EDTA 10 minutes at 37° C. then the reaction stopped with HBSS+serum. Cells were triturated to a suspension and plated on 24 well plates coated with matrigel in DMEM (high glucose) supplemented with FCS (5%), N2 (GIBCO) and antibiotics. Drug treatments were supplemented every 12 hours.

Western Blotting

Western blots were performed essentially as described (Bayle, J. H. et al. “Hyperphenylalaninemia and Impaired Glucose Tolerance in Mice Lacking the Bifunctional DcoH Gene.” (2002) Published in J. Biol. Chem. 277(32), 28884-28891). Extracts were prepared in RIPA buffer, protein quantified by Bradford Assay (Biorad) and 10 μg of total protein resolved by SDS-PAGE, transferred to PVDF (Millipore) filters and Pax6 or HSP90 identified with specific antibodies (1:1000, Covance and 1:2500 Transduction Laboratories, respectively) incubated 1 hour in TBS+10% nonfat milk. Secondary conjugated to horse radish peroxidase were purchased from Jackson immunochemicals and blots were developed by chemiluminescence (Amersham).

Embryo Culture

The date of observation of a vaginal plug was counted as embryonic day 0.5 and the stage of gestation was confirmed by ultrasound ( Chang, C. P. et al. “Sonographic Staging of the Developmental Status of Mouse Embryos in Utero.” (2003) Published in Genesis 36, 7-11). Uterine horns were removed from sacrificed mothers to a petri dish containing Hank's Balanced Salt Solution (HBSS), fat was removed and the uterus cut up to isolate embryos. Individual deciduum were carefully removed from the uterus by carefully pulling apart the muscular layers in a fresh petri dish containing HBSS (sometimes supplemented with 2 mg/mL glucose) under a dissecting microscope. Embryos were removed by cutting along the lateral axis of the deciduum with fine forceps taking care not to puncture the embryonic membranes and teasing off the decidual layers overlying the embryo. Embryos were then separated from the remaining decidua at the ectoplacental cone or primitive placenta (depending on the age of the embryo).

Reichert's membrane was grasped without the underlying visceral yolk sac, carefully torn away from the embryo and trimmed back to the placenta without ripping the yolk sac. Embryos were incubated in 1 mL culture medium (97% rat serum (Charles River Labs, Boston, Mass., or Harlan Biosciences, Cincinnatti, OH), 2 mg/mL glucose, 100U penicillin G, 100 μg/mL, 250 ng/mL Amphotericin B) in a 2 dram glass vial placed within a sealed 1L plastic bottle that is periodically (8 hours) gassed with 20% oxygen, 5% carbon dioxide, 75% nitrogen (20/5/75) for e8.5, 70/5/20 for e9.5 and 95/5 for e10.5. The bottle rotated slowly in a roller bottle chamber at 37° C. Drugs were added directly to the culture media at the commencement of the culture.

Neurofilament Staining

Embryos were fixed overnight in 4% paraformaldehyde/PBS then dehydrated through a staged ethanol series. Endogenous peroxidases were inactivated with 5% H2O2 for 4 hours in ethanol and the embryos were rehydrated. Embryos were blocked with PBS+3% non fat milk+0.1% Triton X-100+1 mM EDTA for 2 hours and were incubated 24-48 hours with 1:100 anti-neurofilament (2H3, Dev Studies Hybridoma Bank, Iowa City). Embryos were washed in block 6 hours with 1 hour changes and incubated overnight at 4° C. with HSP conjugated anti-mouse secondary. Embryos were washed with PBS+0.1% Triton X-100 4 times for 1 hour and developed with the DAB peroxidase kit from Vector laboratories.

Immunohistology

Embryos were protected with a staged series to 30% sucrose in PBS after fixation in 4% PFA (60 minutes to overnight at 4° C.). Protected embryos were embedded in OCT (VWR) frozen and sectioned on a Leica cryostat to 10 μm. Thin sections were stained according to standard protocols (obtained upon request). Secondary antibodies were purchased from Molecular Probes (Eugene, Oreg.) and primary antibodies to neurofilament, Lim3, Nkx2.2, Pax6, En1, Isl1 were obtained from Dev. Studies. Additional antibodies were from Covance, Zymed or a generous gift of Tadashi Nomura and Noriko Osumi (Sendai, Japan). Immunofluorescence was visualized by confocal microscopy.

Chemistry

NMR spectra were recorded on Varian UI-500 (500 MHz for 1H, 125 MHz for 13C) and AM-400 (400 MHz for 1H, 100 MHz for 13C) spectrometers. Chemical shifts are reported in ppm from tetramethylsilane using the solvent resonance as an internal standard (dimethylsulfoxide, 2.49 ppm in 1H NMR and 39.5 ppm in 13C NMR). Infrared spectra were recorded on a Perkin Elmer Spectrum BX FTIR System. Mass spectral data was acquired at the Stanford University Vincent Coates Foundation Mass Spectrometry Laboratory. Dichloromethane was distilled over calcium hydride prior to use. All other materials were used without purification. The syntheses of C16-alkoxy rapamycin analogs followed the method of Luengo, J. L. et al. “Manipulation of the Rapamycin Effector Domain. Selective Nucleophilic Substitution of the C₇ Methoxy Group.” (1994) Published in J. Org. Chem. 59, 6512-6513.

C16-(S)-3-Methylindole rapamycin. To a flame dried and argon cooled 25-mL round bottom flask was added rapamycin (50.3 mg, 55.0 μmol), 3-methylindole (14.4 mg, 110.0 μmol), and CH2C12 (5.0 mL). This solution was cooled to −40° C. and trifluoroacetic acid (17.0 μL, 220.0 μmol) was added. After 5 h at −40° C., 8 mL ethyl acetate and 8 mL brine were added. The solution was warmed to room temperature, the layers separated, and the organic layer dried with sodium sulfate. After concentration under reduced pressure, the crude material was chromatographed using a mobile phase gradient of 1:1 hexanes/ethyl actetate to 2:3 hexanes/ethyl acetate to yield 55.1 mg (99%) of the desired product. Purity was demonstrated by HPLC using a methanol/water/acetonitrile (36:13:1) mobile phase, a Waters XTerra phenyl 5 mm column at 50° C., and monitoring at 278 nm. The C16-(S) stereochemistry was assigned by comparison of the C22 chemical shift in the 1H NMR spectra of the product with rapamycin and C16-(R)-trimethoxyphenyl rapamycin.i,ii Only the trans-conformer is described in the 1H NMR characterization. IR (film) 3418 s, 2931 m, 1717 s, 1652 s, 1456 m, 1386 w, 990 w, 737m. 1H NMR (400 MHz, DMSO-d6, 25° C.) 10.03 (s, 1H ), 7.36 (d, 1H , J=8 Hz), 7.25 (d, 1H, J=8 Hz), 6.98 (t, 1H, J=7 Hz), 6.91 (t, 1H, J=7 Hz), 6.44-6.08 (m, 5H), 5.43 (dd, 1H, J=10, 15 Hz), 5.25 (d, 1H, J=4 Hz), 5.20 (d, 1H, J=10 Hz), 5.05 (m, 1H), 4.91 (d, 1H, J=6 Hz), 4.59 (d, 1H, J=4 Hz), 4.09 (m, 1H), 3.98 (d, 1H, J=4 Hz), 3.66 (m, 1H), 3.32-3.29 (m, 7H), 3.18-3.16 (m, 3H), 2.92-2.78 (m, 2H), 2.46-2.38 (m, 2H), 2.26-2.06 (m, 3H), 2.14 (s, 3H), 2.04-1.50 (m, 13H), 1.46-1.06 (m, 12H), 1.04-0.80 (m, 15H), 0.74-0.56 (m, 4H). 13C NMR (125 MHz, DMSO-d6, 25° C.) d 210.4, 208.1, 199.0, 169.2, 167.1, 140.3, 138.1, 137.2, 135.4, 135.2, 130.8, 129.0, 128.4, 125.1, 122.7, 120.1, 117.9, 117.5, 110.5, 106.6, 98.9, 85.6, 83.8, 75.6, 73.3, 73.3, 66.9, 57.0, 56.8, 51.1, 44.8, 43.6, 38.5, 38.1, 35.3, 34.9, 33.5, 32.9, 32.6, 31.5, 31.4, 29.0, 26.6, 26.1, 24.5, 21.8, 20.4, 17.4, 16.0, 15.5, 14.7, 13.7, 13.2, 8.6. HRMS calculated for C59H85N2O12 (M+H): 1013.6103 amu, found (ESI) 1013.6096 amu.

Synthesis of C20-Methallylrapamycin is described above. C16-(S)Butylsulfonylrapamycin (AP23050) and C16-(S)7-methylindolerapamycin (AP21987) were generously provided by Ariad pharmaceuticals (Cambridge, Mass.).

Mutagenesis

All FRB mutants were prepared by the Quikchange mutagenesis method (Stratagene) using protocols provided by the manufacturer. Briefly, PCR reactions containing the parent plasmid pBJ5-FRB-VP16-HA or pBJ5-FRB*-VP16-HA and overlapping oligonucleotides containing the relevant base changes were performed with Pfu polymerase (Stratagene, San Diego, Calif.). PCR reactions were digested with the methylation sensitive restriction endonuclease Dpn1 to eliminate the parent plasmid and the reaction was transformed in XL10-Gold Ultracompetent E. coli (Stratagene). All mutants were confirmed by sequencing.

SeAP Assay

Drug efficacy was determined in a three-plasmid transcriptional switch reporter assay. ˜106 COS1 cells were coelectroporated with 2 μg of the DNA binding construct pBJ5-Gal4-FKBP(3), 2 μg pBJ5-VP16 (or the relevant mutant FRB) and 2 μg of the Gal4-SeAP reporter. Electroporation details are available upon request. Cells were immediately aliquoted to 2 or 3×96 well plates with flat bottoms. After 24 hours incubation media was supplemented with a rapalog in triplicate and in serial two fold dilutions. Following 24 hours further incubation, cells with media were wrapped carefully in plastic wrap and heated to 65° C. for two hours to inactivate endogenous phosphatases. 50 μL of media was transferred to a black 96 well plate (Costar 3915) and supplemented with 1 mM methylumbelliferrylphosphate (Sigma, St. Louis, Mo.) in 1M diethanolamine (pH 10.0 with carbonate). After 16 hours incubation at 37° C. SeAP activity was measured by fluorescence with a Spectamax M2 fluorometer (Molecular Devices, Sunnyvale, Calif.) with transmission set at 355 nm and emmission set at 460 nm. Data was analysed with Softmax Pro (Molecular Devices), Microsoft Excel and Prism 3 (GraphPad Software).

Transfections and Staining

COS1 cells were electroporated with 1 μg pS-FKBP-GSK3β-GFP, 1 μg of the ‘exporter’ pBJ5-FRB(TLW)-Exp-HA containing a nuclear export sequence derived from the rev protein of Human Immunodeficiency Virus and 3 μg of the ‘importer’ pBJ5-FRB(KTF)-Imp with a nuclear localization sequence from SV40 Large T antigen. Cells were plated on autoclaved coverslips and incubated 36 hours followed by 1 hour of rapalog treatment (10 nM). Cells were fixed 10 minutes in 4% paraformaldeyde/PBS at 4° C. and mounted for microscopy with DAPI to visualize the nucleus. Microscopy was perfomed with a Leica confocal microscope and Leica software.

A number of different mutant FRBs were prepared and are set forth in the Table provided in FIG. 14. This table indicates the binding affinities for the different rapalogs for the different mutants of FRB. The results show that one can efficiently select which mutant FRB is to be stabilized or allowed to be degraded.

### Results

Synthesized rapamycin analogs having lower toxicity, C16-methallylrapamycin (C16-MaRap) and C20-methallylrapamycin (C20-MaRap) bound FRB* were prepared which did not bind wild type FRB (FIG. 1A). The rapalogues were tested to determine if they could activate transcription by recruitment of chimeric transcriptional activation and DNA binding domains (Ho, S. N., Biggar, S. R., Spencer, D. M., Schreiber, S. L., and Crabtree, G. R. “Dimeric ligands define a role for transcriptional activation domains in reinitiation.” (1996) Published in Nature 382, 822-826; Rivera, V. M., Clackson, T., Natesan, S., Pollock, S., Amara, J. F., Keenan, T., Magari, S. R., Phillips, T., Courage, N. L., Cerasoli, F., Jr., et al. “A system for pharmacologic control of gene expression.” (1996) Published in Nature Medicine 2, 1028-1032). FKBP fused to the GAL4 DNA binding domain and FRB or FRB* fused to the activation domain of VP16 were co-transfected into COS-1 cells along with a GAL4-dependent secreted alkaline phosphatase (SeAP) reporter plasmid. Addition of rapamycin or rapalogues resulted in heterodimerization of the fusions and activation of reporter gene expression. Titration of the molecules on either FRB or FRB*-VP16 transfected cells, showed that C20-MaRap bound FRB with low efficacy (EC50=225.9±15.04 nM), but retained a high activity towards FRB* (EC50=3.10±0.36 nM) (FIG. 1B and C). In contrast, rapamycin was highly effective on both FRB (EC50=0.46±0.03 nM) and FRB* (EC50=0.43±0.03 nM). Like C20-MaRap, C16-MaRap exhibited selective binding for FRB* (EC50=15.92±1.5 nM) versus FRB (EC50=272.9±13.48 nM), but was not as effective as C20-MaRap on either substrate. The results show that C20-MaRap has improved characteristics for selective dimerization of FRB*-fusion proteins to FKBP12 while avoiding the toxic effects of FRAP inhibition.

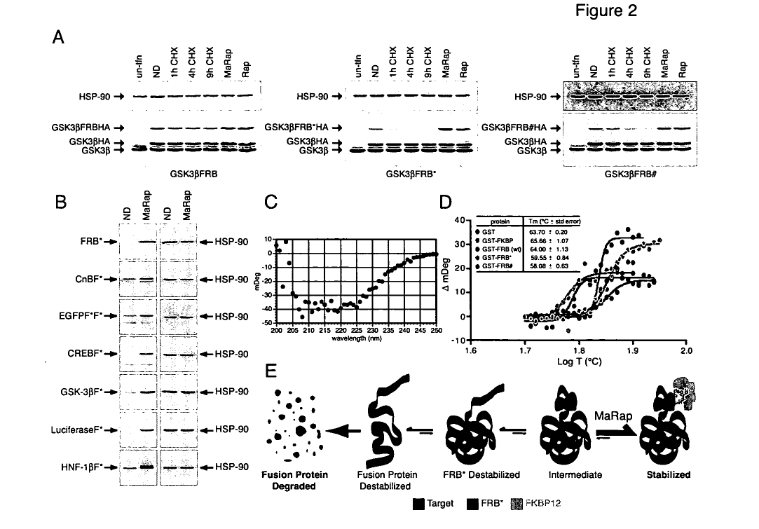

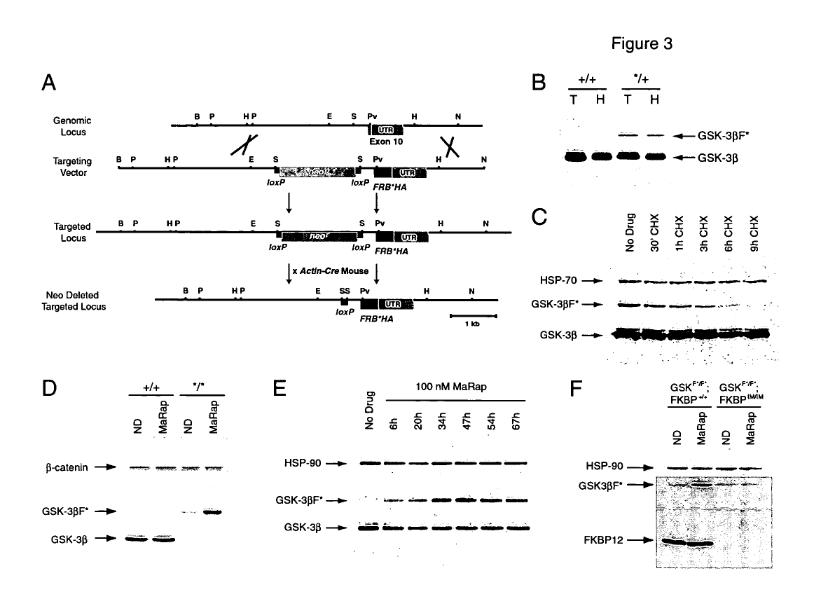

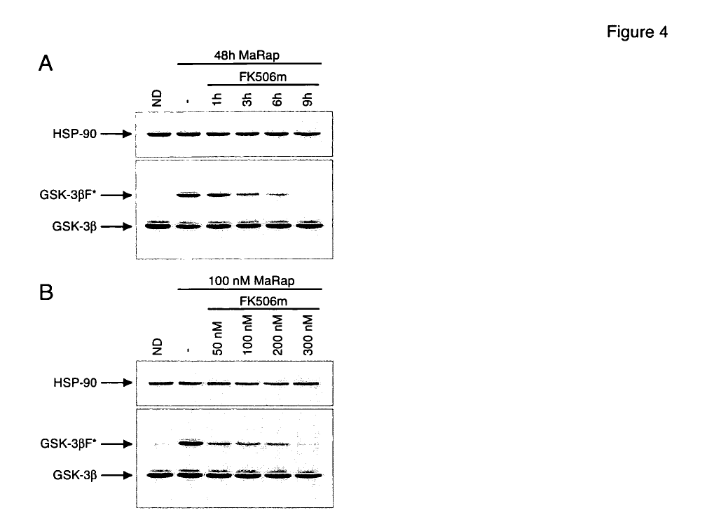

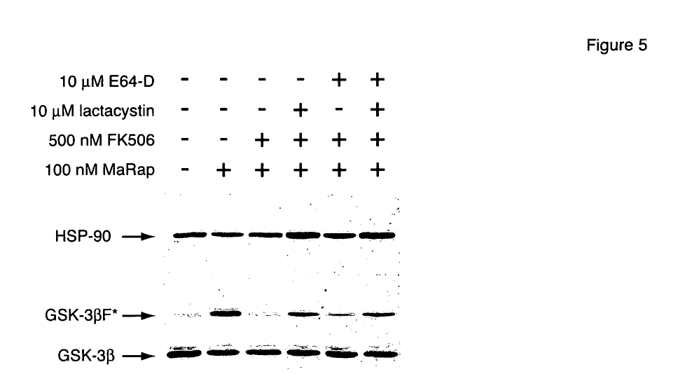

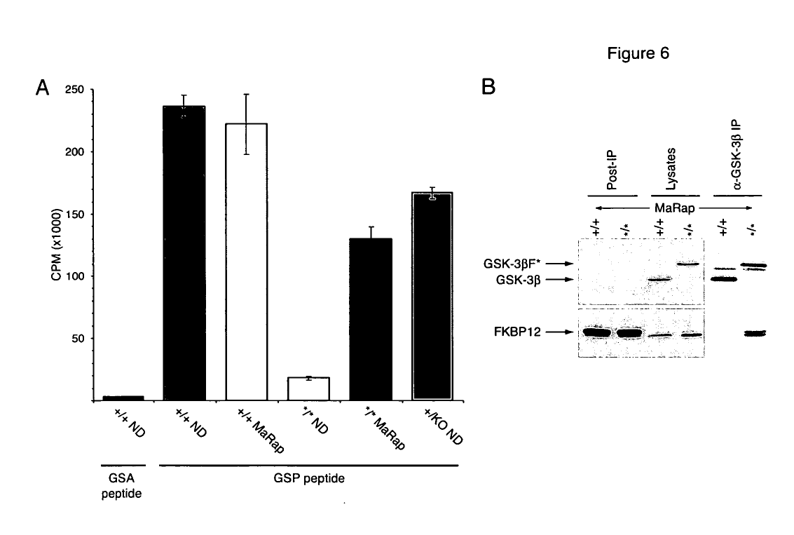

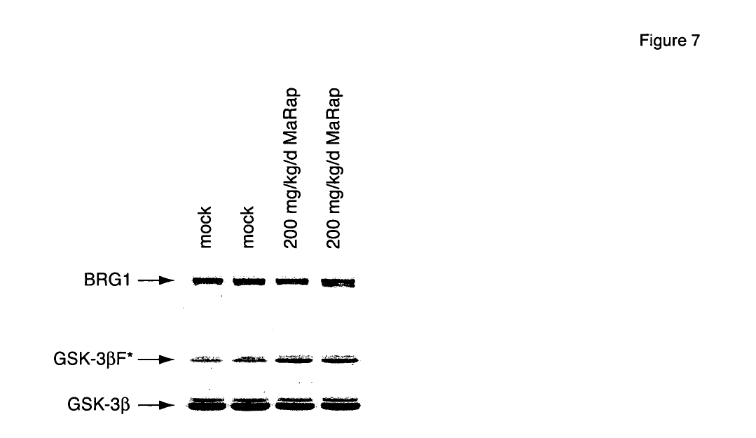

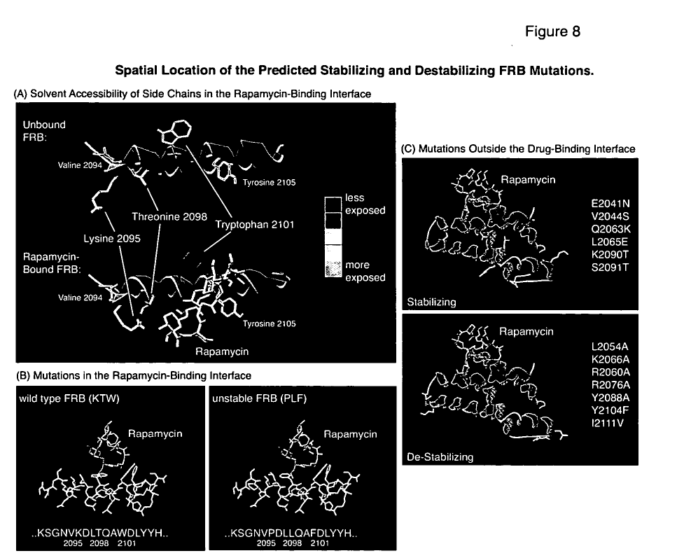

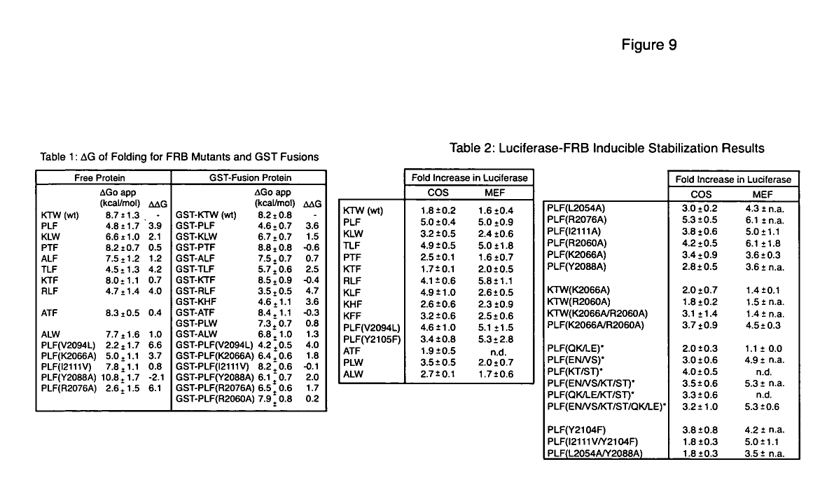

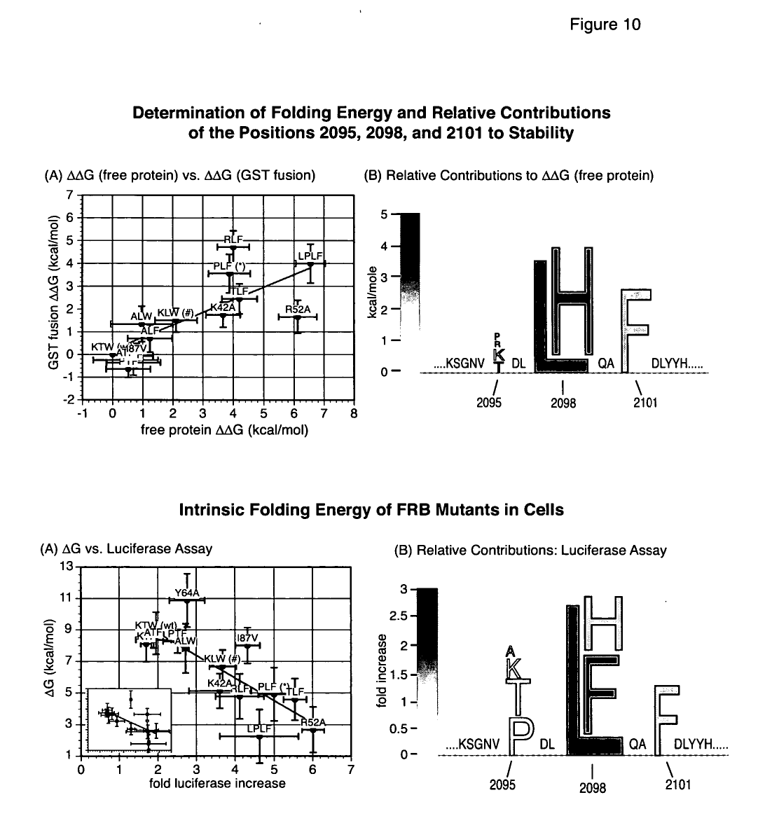

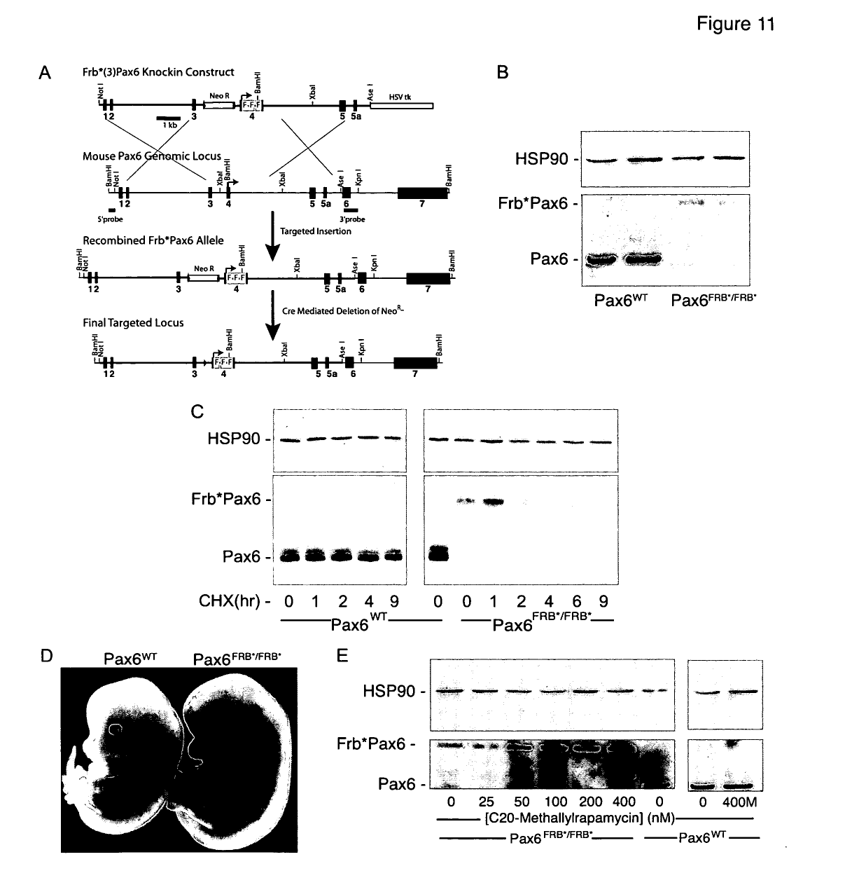

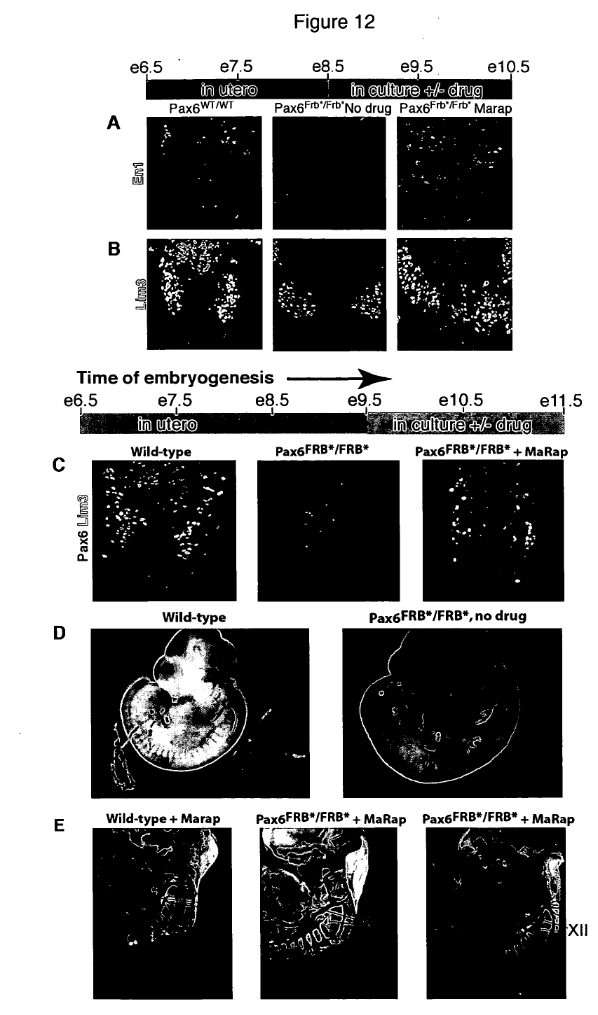

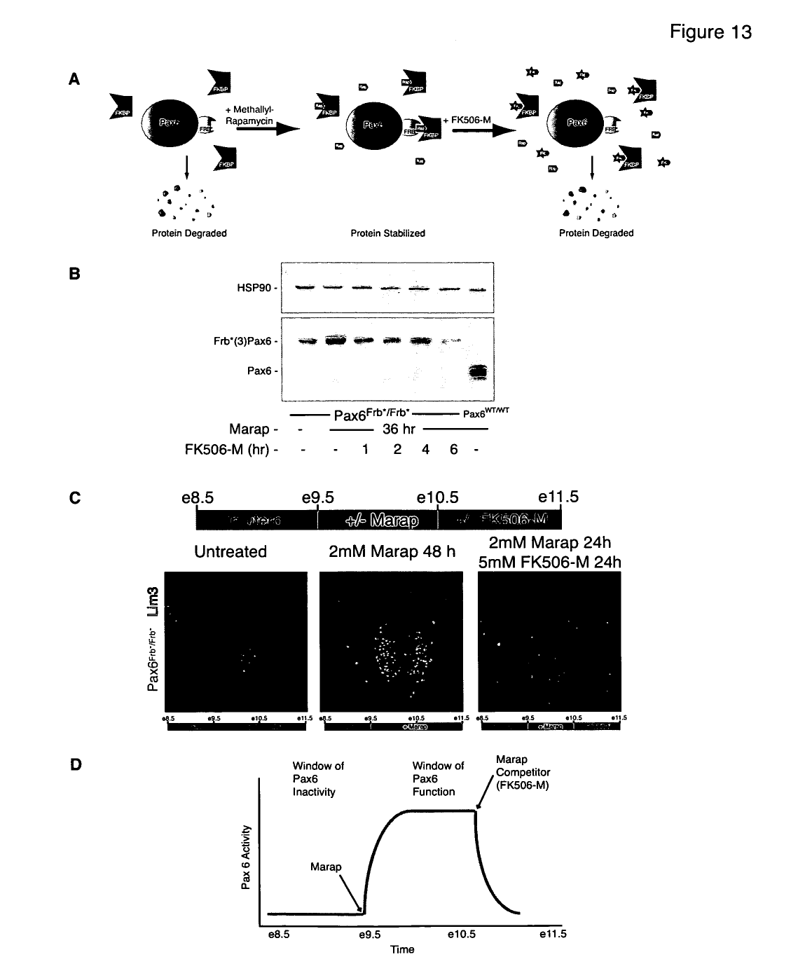

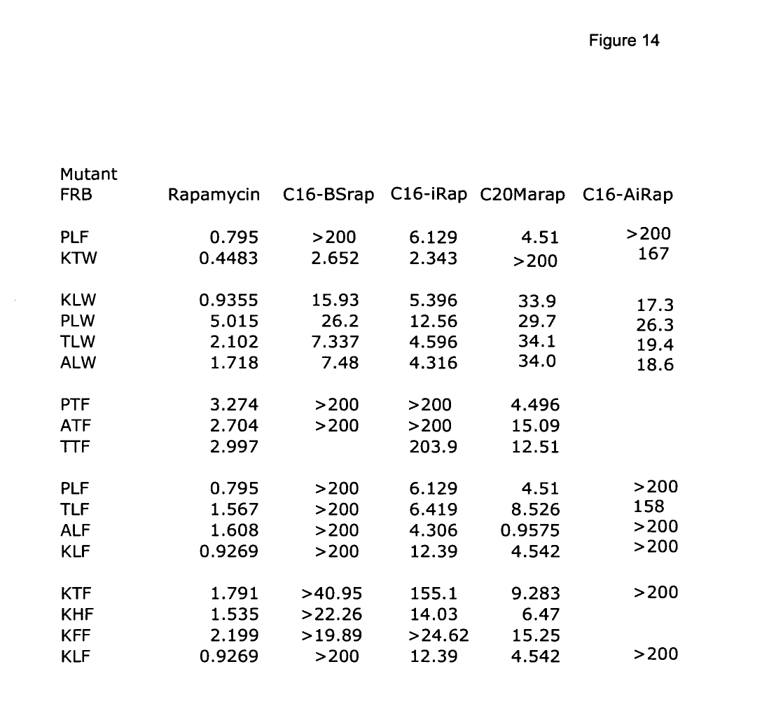

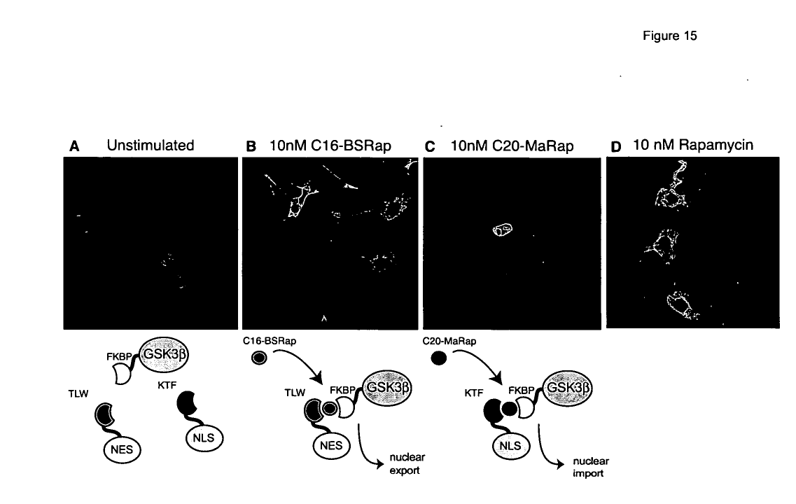

That FRB* directs fusion proteins to degradation was shown as follows. GSK-3β was fused to FRB* in an expression vector to allow the C20-MaRap regulation of overexpressed GSK-3β in transfected cells. The results show that GSK-3β FRB* was expressed at a lower level than an otherwise identical GSK-3β FRB fusion or co-transfected unmodified GSK-3β in transiently transfected mouse embryonic fibroblasts (MEFs) (FIG. 2A). When the transfected cells were treated with the protein translation inhibitor cycloheximide (CHX) it was observed that GSK-3β FRB*, unlike GSK-3β FRB, was rapidly degraded (FIG. 2A). Addition of rapamycin or C20-MaRap restored GSK-3β FRB* expression to normal. Similar results were observed with another fusion protein FRB*-Pax6 fusion protein expressed in CHO cells and with a Luciferase-FRB* fusion expressed in COS-1 cells. These results show that FRB* directs fusion proteins to degradation.

We attribute the major cause of FRB*-mediated instability to the presence of the T2098L mutation. We show this by repeating the GSK-3β assay with FRB# which contains only the T2098L substitution present in FRB* and is otherwise wild-type. GSK-3β FRB#, like GSK-3β FRB*, shows an increased degradation rate relative to GSK-3β FRB, which was also reversed by rapamycin-mediated recruitment to FKBP12 (FIG. 2A).

We investigated whether FRB* conferred similar reversible instability to other fusion proteins by expressing a collection of eight FRB* fusion proteins in MEFs in the presence or absence of C20-MaRap (FIG. 2B). Despite their dissimilarities in molecular weight, subcellular site of action, and structure, the levels of each FRB* fusion protein increased between 3.5 and 100 fold upon drug addition (data not shown). These results suggest that FRB* has the general property of conferring reversible instability to fusion proteins.

The melting temperature (Tm) of FRB, FRB*, and FRB# separately fused to GST was determined by CD at 230 nm, a beta-sheet absorbing wavelength. Results show that GST-FRB* and GST-FRB# had a Tm approximately 5° C. lower than GST fused to wild-type FRB (FIG. 2C). These results further confirm that the T2098L mutation destabilizes FRB structure. Thus FRB* confers two characteristics to fusion proteins, first, it provides the ability to dimerize to FKBP12 in the presence of the non-toxic rapalog, C20-MaRap, and second, it causes the destabilization and resulting degradation of fusion proteins. This directed degradation is blocked by dimerization to FKBP12 using C20-MaRap.

Dimerization may “lock” FRB* in a folded state, energetically stabilized by interactions with FKBP12 and the rapalogue (FIG. 2D). These dual features of FRB* facilitate the creation of the subject conditional allele system, whereby a tagged protein is inducibly stabilized to functional levels only in the presence of a highly specific small stabilizing molecule.

A FRB* knock-in allele of GSK-3β was constructed to permit conditional regulation of GSK-3β at the protein level . This circumvents the difficulties associated with the pleiotrophy of GSK-3β where murine deletion mutants are expected to have complicated phenotypes (Hoeflich, K. P., Luo, J., Rubie, E. A., Tsao, M. S., Jin, O., and Woodgett, J. R. “Requirement for glycogen synthase kinase-3β in cell survival and NF-κB activation.” (2000) Published in Nature 406, 86-90) that would be difficult to deconvolute. A selectable plasmid was constructed with a neomycin resistance selection cassette flanked by loxP sites placed in the last intron of GSK-3β and a FRB*HA sequence placed immediately before the stop codon of GSK-3β (FIG. 3A). Homologous recombination in ES cells was used to target this construct to the endogenous GSK-3β locus. Mice were derived from correctly targeted cells and the neomycin resistance cassette was removed by crossing these mice with mice expressing the Cre recombinase under the ubiquitous β-actin promoter (Lewandoski, M., Meyers, E. N., and Martin, G. R. “Analysis of Fgf8 gene function in vertebrate development.” (1997) Published in Cold Spring Harb. Symp. Quant. Biol. 62, 159-168). This ensures that the only genomic changes in the derived mouse line was a single loxP site in the last intron of GSK-3β and the FRB*HA sequence inserted at the extreme 3′ end of the GSK-3β coding region. This design was employed to ensure minimal disruption of GSK-3β regulatory elements. mRNA analysis confirmed that GSK-3 βFRB* mRNA was present at normal levels. A C-terminal FRB* fusion was chosen to prevent interference with the N-terminal serine-9 phosphorylation site of GSK-3β. The C-terminal tag GSK-3βFRB* retained the ability to inhibit NF-AT-dependent transcription in transient tranfection assays in Jurkat T cells and could still be phosphorylated at serine-9. Although GSK-3β^{+/FRB*} mice were viable and fertile, most GSK-3β^{FRB*/FRB*} mice died immediately following birth, being present at normal Mendelian ratios at embryonic day E18.5. The mice exhibit a complete cleft of the secondary palate. The occasional homozygous pup that did not die immediately after birth failed to suckle and died within 12 hours of birth. This phenotype is indistinguishable from that of GSK-3β^{KO/KO} and GSK-3β^{KO/FRB*} mice on a similar outbred strain background. The expression of GSK-3βFRB* was examined in heterozygous mice, and GSK-3βFRB* levels were found to be greatly reduced relative to wild-type protein in all tissues examined. Heart and thymus expression levels are shown in FIG. 3B. This result is consistent with the FRB*-directed degradation of GSK-3βFRB* seen in transfection experiments and with the severe loss-of-function phenotype of GSK-3β^{FRB*/FRB*} mice.

GSK-3βFRB* is inducibly and reversibly stabilized in MEFs. MEF's were prepared containing the GSK-3β^{FRB*} allele. Heterozygous cells expressed greatly reduced levels of GSK-3βFRB* consistent with its low level of expression in adult tissues. Enhanced degradation of GSK-3βFRB* was observed relative to the wild-type GSK-3β protein when translation was blocked with CHX (FIG. 3C). A 36 hour treatment with C20-MaRap restored GSK-3βFRB* levels in GSK-3β^{FRB*/FRB*} cells to those of GSK-3β in wild-type MEFs (FIG. 3D). Rapamycin produced an identical effect and neither drug had an effect on wild-type GSK-3β, β-catenin, or HSP-90 expression. A time course of C20-MaRap treatment on heterozygous MEFs was performed to study the rate of inducible stabilization, and it was found that stabilization was complete in approximately 24 hours with half maximal stabilization observed at 8-12 hours (FIG. 3E). This stabilization did require FKBP12 binding, as observed by the complete loss of C20-MaRap-induced stabilization in fibroblasts that are doubly homozygous for GSK-3β^{FRB*} and an FKBP12 allele, FKBP12^{IM}, that lacks detectable FKBP12 expression (FIG. 3F). The GSK-3βFRB* induction in MEF's is reversible. FK506 was used which binds to FKBP12 with high affinity to form a complex that interacts with and inhibits the phosphatase calcineurin (Liu, J., Farmer, J. D., Jr., Lane, W. S., Friedman, J., Weissman, I., and Schreiber, S. L. “Calcineurin is a common target of cyclophilin-cyclosporin A and FKBP-FK506 complexes.” (1991) Published in Cell 66, 807-815). FK506 reverses inducible stabilization by competing with C20-MaRap for binding to FKBP12. GSK-3βFRB* was stabilized in heterozygous MEFs and the cells then treated across a time course with monomeric FK506 (FK506M). FK506M is a derivative of FK506 that does not bind to calcineurin but retains the ability to bind FKBP12 (Spencer, D. M., Wandless, T. J., Schreiber, S. L., and Crabtree, G. R. “Controlling signal transduction with synthetic ligands.” (1993) Published in Science 262, 1019-1024). Following FK506M treatment, GSK-3βFRB* was rapidly degraded, returning to steady state low levels in less than 9 hours (FIG. 4A). Inducible stabilization is therefore readily reversible. Different relative concentrations of FK506M to C20-MaRap were used and a gradient produced of steady state GSK-3βFRB* levels (FIG. 4B). The ratio of the two drugs can be used to titer GSK-3βFRB* activity and produce a “chemical allelic series”.

The mechanism underlying FRB*-mediated degradation is directed by the proteasome. The degradation was tested in the presence of specific protease inhibitors. GSK-3βFRB* was stabilized to normal levels in heterozygous MEFs before the stabilization was reversed by the addition of FK506 to the culture media. When the instability was reversed in the presence of lactacystin (FIG. 5) or MG132, two proteasomal inhibitors, GSK-3βFRB* remained stable. E-64D, an inhibitor of lysosomal cysteine proteases, did not protect GSK-3βFRB* from degradation. This shows that FRB* mediated-destabilization results from proteasomal degradation.

GSK-3β activity is also restored by inducible stablilization.

GSK-3β immunoprecipitation-kinase assays were carried out on fibroblast lysates to test whether inducible stabilization restored GSK-3β activity. Immunoprecipitated wild-type GSK-3β effectively incorporated radiolabeled ATP into the GSP peptide from glycogen synthase, but not into the GSA peptide, which contains a serine-alanine mutation at the GSK-3β phosphorylation site (FIG. 6A). Destabilized GSK-3βFRB* isolated from untreated GSK-3β^{FRB*/FRB*} MEFs contained less than 10 percent of normal GSK-3β kinase activity. However, GSK-3βFRB* immunoprecipitated from C20-MaRap treated GSK-3β^{FRB*/FRB*} cells had an over 7-fold increase in activity and approximately 60% of the activity of wild-type cells. Furthermore, the stabilized GSK-3βFRB* remained bound to FKBP12 during the kinase assay, indicating that FKBP12-C20-MaRap binding does not inhibit GSK-3β's activity or substrate access (FIG. 6B). GSK-3βFRB* stabilized in GSK-3β^{FRB*/FRB*} MEFs had a similar activity to that present in identically prepared and treated GSK-3β^{+/KO} MEFs (FIG. 6A). Since heterozygous GSK-3β^{KO} cells and mice appear completely normal, we anticipate that inducible stabilization could recover sufficient levels of GSK-3β activity to restore the kinase's normal functions. To determine if C20-MaRap could be delivered to embryos, E10.0 pregnant heterozygous GSK-3βFRB* mice (crossed to heterozygous male), were injected with 200 mg/kg/day of C20-MaRap. The injection course consisted of 3 IP injections repeated every 12 hours, followed by the harvesting of the embryos at E11.5. Neither the mother nor the embryos were overtly affected by this drug course. In contrast, rapamycin, similarly injected at 10 mg/kg/day, produced “flat-top” embryos due to an E16 failure of telecephalon outgrowth, demonstrating the necessity of using non-teratogenic rapalogues. Protein lysates were prepared from the C20-MaRap treated embryos and from stage-matched embryos from a mock-injected mother, and analyzed by Western blotting. GSK-3βFRB* protein levels were partially restored in embryos from the C20-MaRap-treated mothers (FIG. 7).

Mutational Analysis

An important feature for the subject invention is that binding to rapamycin must compensate for the instability introduced by the mutations; thereby placing protein stability under control of the small molecule. To address this, we examined the structure of the wild type FRB domain both in the absence of rapamycin and bound to the drug. Our initial focus was on the three amino acids (2095, 2098, and 2101) known to provide drug-dependent stability in the PLF(FRB*) mutant. One striking feature of these residues is that, upon binding, the accessibility of the side chains is altered. Specifically, in the bound structure, all three positions become less solvent exposed. This change is particularly dramatic for the lysine and threonine residues. For example, in the unstable FRB, PLF, the threonine residue is replaced by a leucine. We then introduced more hydrophobic side chain at these sites. A mutation from valine to leucine at amino acid 2094 was introduced. This mutant is termed PLF(V2094L). Similarly, a tyrosine to phenylalanine mutation was created at 2105 and this mutant is termed PLF(Y2105F). ROSETTA is an automated tool for virtual mutational analysis that has been described in detail elsewhere (Kim, D. E., Chivian, D. & Baker, D. “Protein Structure Prediction and Analysis Using the Robetta Server.” (2004) Published in Nuc. Acids Res. 32, W526-W531). Using the crystal structure of wild type KTW in complex with rapamycin and FKBP as the primary data set, two types of calculations were performed. One set restricted virtual mutagenesis to amino acids within the rapamycin-binding interface. Specifically, we explored the sites (2095, 2098, and 2101) that, in PLF, are known to provide drug-dependent stability. At these sites, Monte Carlo iterations varied the identity of the amino acid residues and sampled all common side-chain rotamers. Backbone alignments were constrained according to the structural coordinates. Output data was in the form of tables of predicted intrinsic folding free energy (ΔG) for each mutational outcome. From the resulting data, mutations were selected that predict substantial positive or negative changes in ΔG.

To test the effects of mutations on stability, we first cloned KTW(FRBwt) and PLF, purified the proteins and determined their ΔG in vitro by monitoring tryptophan fluorescence during chemical denaturation (FIG. 9). Using this method, we confirmed that PLF is unstable relative to KTW. Specifically, the three PLF mutations had a ΔΔG of 3.9 kcal/mole. To explore the effects of additional mutations, the ΔG values for the remaining mutants were determined. For mutations outside the drug-binding site, these changes were made in the context of either KTW or PLF (i.e. PLF(R2076A) has the three PLF mutations and the R2076A change). As shown in Table 1, these mutations produced FRB domains that vary in intrinsic folding energy over a range of approximately 6-7 kcal/mol.

To test the effects of mutations on stability, we first cloned KTW and PLF, purified the proteins and determined their ΔG in vitro by monitoring tryptophan fluorescence during chemical denaturation (FIG. 9). Using this method, we confirmed that PLF is unstable relative to KTW. Specifically, the three PLF mutations had a ΔΔG of 3.9 kcal/mole. To explore the effects of additional mutations, the ΔG values for the remaining mutants were determined. For mutations outside the drug-binding site, these changes were made in the context of either KTW or PLF (i.e. PLF(R2076A) has the three PLF mutations and the R2076A change). As shown in Table 1, these mutations produced FRB domains that vary in intrinsic folding energy over a range of approximately 6-7 kcal/mol.

When unstable FRB tags are fused to proteins, it is important that they disrupt the stability of the entire fusion. To test this possibility for the set of FRB mutants, the ΔG of FRB-GST fusions was determined (FIG. 9). These results indicate that mutations in FRB yield fusions that, similar to the free domains, vary in ΔG. The range of ΔΔG observed for the fusion proteins was less than 5 kcal/mole, compared to more than 6 kcal/mol for the free FRB domains. This result suggests that the fusion protein dampens the effects of tag instability and modulates the impact of mutations on the overall free energy. To further explore this relationship, we compared the ΔΔG of the free FRB domains and the corresponding FRB-GST fusions (FIG. 10). A linear correlative relationship was observed between these values, which suggests that the relative stability of the FRB tag is reliably and directly replicated as part of the GST fusion. These results suggest that the ΔG of the fusion is partially determined by the stability of the tag.

Greater insight into the origins of FRB stability might allow rational design and implementation of conditional tags. Among the important issues are whether all sites contribute equally to stability and which side chains provide stable and unstable tags. Using the series of mutations generated in the rapamycin-binding interface, we explored the relative contributions of each mutation to the ΔG (FIG. 10). Surprisingly, a majority (55%) of stability was dependent on the identity of amino acid 2098 at the central site. The third position was the next best contributor to stability (40%) and the first position was less important (5%). Although only a subset of amino acid changes were performed at a chosen few sites on FRB, these results help explain the origins of PLF's instability. These results also point to methods for controlling stability in this domain by identifying amino acid substitutions that can be used to enhance or decrease relative stability. While measuring the stability of purified proteins in vitro gives useful information, biological applications require that observed trends be replicated in the complex environment of a cell. Therefore, we sought to develop a rapid assay for screening FRB stability in vivo. We hypothesized that a FRB fusion to firefly luciferase (Luc) may supply a reagent for this purpose. In this scenario, enzymatic activity would be low in the absence of rapamycin but protein levels and luminescence would increase with addition of drug. To determine the effect of tag stability on protein's activity in cells, we generated luciferase fusions to the set of FRB mutations and screened them using the luciferase assay. In these experiments, the luciferase activity of cells treated with 5 nM rapamycin was compared to untreated controls and the constructs were tested in both COS1 cells and mouse embryonic fibroblasts (MEFs) to explore potential cell-to-cell variations. When fused to luciferase, these tags provided an array of proteins with variable sensitivity to rapamycin (FIG. 9). Together with the in vitro data, these results suggest that degradation in both COS1 cells and MEFs is influenced by the relative free energy of the FRB mutants (FIG. 10).

Similar to the analysis performed on the ΔG measurements in vitro, relative contributions to stability were calculated using the luciferase values (FIG. 10). Despite the differences between the experimental parameters employed, similar trends were observed in vivo and in vitro. Namely, mutation of all three sites in the drug-binding interface of FRB was required for full destabilization of fusions; however, the second position, 2098, is the principle contributor to drug-recoverable stability. These results support a hypothesis in which changes to the primary sequence of FRB that alter the intrinsic folding energy of the domain cause concomitant changes in the stability and degradation of FRB fusions in cells.

We have generated a series of mutations in the FRB domain and determined their stability both as independent domains and in the context of two different fusion proteins. In general, the ΔG of the FRB mutant alone was a good predictor of the stability of the fusion protein, both in vitro and in cells. Thus, we suspect that the intrinsic stability of the target protein may influence the extent to which the tag can destabilize the fusion. More stable targets may require more unstable tags for full degradation. For example, we have reported PLF fusions to 8 different targets that vary in size and structure (Stankunas, K., Bayle, J. H., Gestwicki, J. E., et al. “Conditional Protein Alleles Using Knockin Mice and a Chemical Inducer of Dimerization.” (2003) Published in Mol. Cell 12, 1615-1624). In each case, protein levels are low in the absence of rapamcyin and can be restored with drug. However, the degree of induction ranges from approximately 2-5 fold to over 100-fold. Although the parameters that determine these differences are not yet clear, these results suggest that structural or physical properties of the target influence their behavior in inducible stabilization experiments. The availability of a series of FRB tags with variable intrinsic stability, which can be matched to a specific requirement for ΔG, may be useful for application to emerging targets.

One goal achieved in this study was to locate the limits of FRB stability. Using mutations in FRB, we were able to introduce an additional 2 kcal/mol of instability beyond that provided by the PLF mutations (with the mutations R2076A and PLF(V2094L)). At the other extreme, few mutations were uncovered that dramatically improved stability of the domain beyond the wild type KTW. In general, the majority (13 of 15) of FRB mutants could be grossly characterized as being either stable (ΔΔG between 0 and 1.2 kcal/mol) or unstable (ΔΔG between 3.7 and 6.6 kcal/mol). The apparent upper limit on instability might arise because proteins with additional instability fail to express at appreciable levels. Thus, the free energy range of the FRBs presented here may represent natural or technical limits. We show that these variants are a representative series that can be used to generate fusions for inducible stabilization experiments.

Inducible Stabilization of Pax6

The subject system was employed to study the functions of the Pax6 transcription factor during murine embryogenesis. Pax6 is a critical regulator of neural development as well as the development of the eye and nose ( Grindley, J. C. et al. “The Role of Pax-6 in Eye and Nasal Development.” (1995) Published in Development 121(5), 1433-1442; Ashery-Paden, R. et al. “Pax6 Lights-Up the Way for Eye Development.” (2001) Published in Curr. Opin. Cell Biol. 13(6), 706-714). Mutations in FRB*(PLF) that give specificity for MaRap, destabilize the structural integrity of the FRB* domain and targets fused to FRB* when expressed at physiological levels ( K. Stankunas et al., Mol Cell 12, 1615 (Dec, 2003)). To conditionally regulate Pax6 function during development we have produced a line of mice in which three copies of a FRB* minigene were fused to the 5′end of the Pax6 gene by targeted recombination in ES cells (FIG. 11A). The protein produced from this allele, FRB*Pax6, was expressed at far lower levels than wild-type Pax6 in cortical neural cultures derived from e13.5 embryos of Pax6^{FRB*/FRB*} mice (FIG. 11B). The translation inhibitor cycloheximide was added to similar cortical cultures to examine the kinetics of Pax6 destruction. FRB*Pax6 protein had a markedly increased rate of turnover relative to wild-type Pax6, which was stable over the 9 hour timecourse (FIG. 11C).

The instability of FRB*Pax6 protein produces a semi-dominant phenotype in Pax6^{FRB*/FRB*} mice closely similar to that of the Pax6 null mutant, Small eye. Pax6^{FRB*/+} adult mice are viable and fertile, but have micropthalmia of varying expressivity with cataracts. Pax6^{FRB*/FRB*} mice have difficulty breathing, do not suckle and die at or shortly after birth with no eye or nose (FIG. 11D). Pax6^{FRB*/FRB*} embryos do not form lens or nasal placodes and undergo subsequent invaginations. The nasal cavities and the lens and optic vesicles fail to develop with the optic vesicle appearing to form a Pax2 expressing optic stalk before receding. The embryonic brain lacks olfactory bulbs and has a thinner dorsal telencephalon.

Our previous study demonstrated that recruitment of FKBP12-MaRap to FRB* stabilizes the integrity of FRB* and increases the expression of FRB*-tagged proteins ( Stankunas, K. et al. “Conditional Protein Alleles Using Knockin Mice and a Chemical Inducer of Dimerization.” (2003) Published in Mol. Cell 12, 1615-1624). Addition of increasing quantities of C20-MaRap to cortical neural cultures derived from Pax6^{FRB*/FRB*} embryos produced a dose-dependent increase in FRB*Pax6 protein to a level similar to that of wild-type Pax6 (FIG. 11E). MaRap treatment had no effect on wild-type Pax6 expression. FRB*Pax6 was stabilized between 24 and 36 hours of drug treatment (data not shown). The kinetics of FRB*Pax6 turnover were markedly slowed in the presence of C20-MaRap (data not shown) indicating that the induction of FRB*Pax6 expression by MaRap was the result of stabilization of FRB*Pax6. To characterize time windows of Pax6 function, we applied inducible stabilization of FRB*Pax6 to whole embryo cultures of litters derived from Pax6^{FRB*/+} parents. Embryos developed normally for 48 hours starting at embryonic day 8.5 (e8.5) or e9.5 (data not shown). Addition of MaRap to embryo cultures had no obvious effect on wild-type embryos, but the expression of FRB*Pax6 protein in the spinal cord of P Pax6^{FRB*/FRB*} embryos was rescued (data not shown). Detailed analysis of the expression pattern of FRB*Pax6 in sections of the developing nervous system indicated that FRB*Pax6 protein was inducibly stabilized only in the tissues that normally express Pax6 protein. The faithful expression profile of the latent protein illustrates the principal advantage of a knockin strategy to introduce the conditional allele into the genome.

We examined the effect of stabilization of FRB*Pax6 on the patterning of the caudal neural tube. In the developing spinal cord, Pax6 is expressed in the ventricular zone exclusively in neural progenitor cells (Ericson, J. et al. “Pax6 Controls Progenitor Cell Identity and Neuronal Fate in Response to Graded Shh Signaling.” (1997) Published in Cell 90(1), 169-180; Osumi, N. et al. “Pax-6 is Involved in the Specification of Hindbrain Motor Neuron Subtype.” (1997) Published in Development 124(15), 2961-2972; Stoykova, A. et al. “Forebrain Patterning Defects in Small eye Mutant Mice.” (1996) Development 122(11), 3453-3465). Pax6 is a member of an array of homeodomain containing transcription factors that impart spatial identity along the dorsal-ventral axis of the ventral neural tube in response to a gradient of the morphogen Sonic hedgehog (Shh) secreted from the ventral floor plate. To confirm that stabilized FRB*Pax6 can perform Pax6-dependent activities we examined the expression of Engrailed1 (En1) in V1 interneurons of the developing spinal cord. Differentiation of V1 interneurons depends on Pax6 function (17). MaRap treatment of e8.5 embryos cultured for 48 hours to about 30 pairs of somites rescued the development of En1 expressing cells in the V1 domain of Pax6^{FRB*/FRB*} spinal cord (FIG. 12A). Similarly, stabilization of FRB*Pax6 produced an expansion of cells expressing Lim3, a marker of somatic motoneurons and V3 interneurons (FIG. 12B).

In the developing embryonic caudal (R6-R8) hindbrain, Pax6 participates in the subtype differentiation of motoneurons. Visceral motoneurons are marked with Isl1 and Phox2b and are born in the most ventral domain of the motor neuron progenitor pool expressing Nkx2.2 (Briscoe, J. et al. “Homeobox Gene Nkx2.2 and Specification of Neuronal Identity by Graded Sonic Hedgehog Signaling.” (1999) Published in Nature 398, 622-627). Differentiated visceral motoneurons migrate to a dorsal position and extend axons to form the vagus (X) nerve. Somatic motor neurons are Pax6 dependent and derived from progenitors specified in a dorsal domain of the motoneuron progenitor pool (Ericson, J. et al. “Pax6 Controls Progenitor Cell Identity and Neuronal Fate in Response to Graded Shh Signaling.” (1997) Published in Cell 90(1), 169-180; Osumi, N. et al. “Pax-6 is Involved in the Specification of Hindbrain Motor Neuron Subtype.” (1997) Published in Development 124(15), 2961-2972; Thaler, J. P. et al. “LIM Factor Lhx3 Contributes to the Specification of Motor Neuron and Interneuron Identity Through Cell-Type-Specific Protein-Protein Interactions.” (2002) Published in Cell 110(2), 237-249; Takahashi, M. et al. “Pax6 Regulates Specification of Ventral Neurone Subtypes in the Hindbrain by Establishing Progenitor Domains.” (2002) Published in Development 129(6), 1327-1338). These cells are marked with Lim3/Lhx3, Mnr2 and Is12. Brightly Lim3 immunopositive motor neurons migrate laterally from the ventricular progenitor one (FIG. 12C) and remain ventrally positioned following differentiation. Wild-type embryos cultured for two days starting at e9.5 also had weakly Lim3 positive cells in the progenitor zone, and some were also Pax6 positive. In the caudal hindbrain of cultured Pax6^{FRB*/FRB*} embryos only the weakly Lim3 positive progenitors were evident, and few if any Lim3 positive cells were in the marginal zone. This phenotype could be rescued with 48 hours of MaRap treatment from e9.5 to e11.5(FIG. 12C). The hypoglossal (XII) nerve, which is derived from Lim3 positive cells, is absent in Pax6 mutant mice (FIG. 12D). Whole mount staining for neurofilament revealed that the production of the fasciculated hypoglossal nerve erupts from the Pax6^{FRB*/FRB*} hindbrain with 48 hours of MaRap treatment from e9.5 to e11.5(FIG. 12E). The expressivity of this rescue is incomplete as the bundled fascicles evident in the hindbrain were not observed to track over the heart to innervate the tongue. Occasionally the nerve fibres tracked without a discernable pattern. These findings may be a result of the mispositioning of their cell bodies on the D-V axis of the hindbrain due to the derepression of Nkx2.2 expression dorsally prior to the induced stablilization of Pax6 and concomitant repression of ventral Pax6 transcription (data not shown).

The experimental modulation of FRB*Pax6 protein expression permitted addressing the kinetics of the requirement for Pax6 for the differentiation of somatic motor neurons. Stabilization of FRB* tagged targets can be blocked and reversed by competition with saturating quantities of an FKBP ligand that does not interact with the FRB domain (FIG. 13A), (Stankunas, K. et al. “Conditional Protein Alleles Using Knockin Mice and a Chemical Inducer of Dimerization.” (2003) Published in Mol. Cell 12, 1615-1624; Spencer, D. M. et al. “Controlling Signal Transduction with Synthetic Ligands.” (1993) Published in Science 262, 1019-1024). FK506M is a derivative of the immunosuppresant FKBP ligand FK506 that does not interact with the FRB of mTOR (or Calcineurin) and is thereby non-toxic (Stankunas, K. et al. “Conditional Protein Alleles Using Knockin Mice and a Chemical Inducer of Dimerization.” (2003) Published in Mol. Cell 12, 1615-1624; Spencer, D. M. et al. “Controlling Signal Transduction with Synthetic Ligands.” (1993) Published in Science 262, 1019-1024). Replacement of MaRap after 36 hours with FK506M on Pax6^{FRB*/FRB*} cortical cultures caused stabilized FRB*Pax6 levels to drop to the basal level within 6 hours (FIG. 13B). We addressed the effect of transient stabilization of FRB*Pax6 on the differentiation of somatic motor neurons in cultured e9.5 Pax6^{FRB*/FRB*} embryos (FIG. 13C). Stabilization for 24 hours with MaRap followed by replacement of MaRap with FK506M resulted in the loss of most of the FRB*Pax6 immunoreactivity except in a small intermediate (D-V axis) domain where FRB*Pax6 levels appears to persist. The basis for this local persistence is unknown. Progenitor cells that are weakly Lim3-positive are evident in reasonably large numbers yet the brightly expressing Lim3-positive somatic motor neurons fail to differentiate in contrast to like embryos treated for a full 48 hours with MaRap. This finding suggests that Pax6function is required continuously from e9.5 past e10.5 for the proper differentiation of somatic motor neurons.

These studies demonstrate several of the potential applications for the rapid and reversible stabilization of FRB* tagged proteins by chemical inducers of dimerization in vivo. The rapid kinetics of induction and destruction make possible the definition of execution points with specificity only for the target protein. The reversibility of the technology makes the function of the protein experimentally controlled over defined temporal windows of development (FIG. 13D). For example, we defined that Pax6 function is required continuously from e9.5 to e11.5 to support the development of somatic motor neurons of the hindbrain. This controlled nature of the system gives it the potential to greatly aid the analysis of complex systemic phenotypes as being a direct effect of the loss of a targeted protein versus an indirect defect in one tissue due to an early loss influencing subsequent development of another tissue. Continued development of this system will permit its application to the analysis phenotypes in disease models of adult animals especially those for which protein stablilization during periods of development that would otherwise result in embryonic lethality can be rescued.

Orthogonal Conditional Protein Alleles

The rapid and reversible stablilization of FRB* tagged proteins has the potential to be a powerful conditional allele system applicable to broad classes of cytosolic proteins (Stankunas, K. et al. “Conditional Protein Alleles Using Knockin Mice and a Chemical Inducer of Dimerization.” (2003) Published in Mol. Cell 12, 1615-1624). To improve the technology for in vivo studies, we sought to understand the basis for the remarkable specificity of MaRap for FRB* (Kd=3.1 nM) over the wild-type FRB (Kd>200 nM). The minimal FRB domain comprises amino acids 2025-2114 of human mTor. FRB* is a triple mutant resulting in the following amino acid substitutions in the 89 amino acid four helical FRB bundle: Lys2095 to Pro, Thr2098 to Leu, and Trp2101 to Phe. We have sought to determine the contribution of each of the three mutations in FRB* to the specificity achieved for C20-Marap. We screened the new variant FRBs for against a library of rapalogs to find new drug mutant combinations that may have improved specificity or pharmacokinetics. As a result we have built an array of FRB mutants that have overlapping specificities for rapalogs derived at C20 or C16. The discrete specificity of individual drug-mutant combinations permits the development of dimerizer strategies that operate in an orthogonal manner in the same cell environment. C20-MaRap interacts poorly with the wild-type FRB(KTW) (EC50>200 nM) yet it binds efficiently with the triple mutant FRB*(PLF). Binding of rapamycin and C20-MaRap was assesed in a rapalog dependent transcriptional switch in which three copies of human FKBP12 were fused to the yeast GAL4 DNA binding domain and the mutant (or wild-type) FRB domain was fused to the potent transcriptional activation domain from Herpes Simplex Virus VP16 protein (Ho, S. N. et al. “Dimeric Ligands Define a Role for Transcriptional Activation Domains in Reinitiation.” (1996) Published in Nature 382, 822-826). Co-expression of these recombinant constructs in COS1 cells with a secreted bacterial alkaline phosphatase (SeAP) reporter containing Gal4 specific DNA binding elements promotes SeAP expression in the only in the presence of a rapalog capable of interacting with FKBP and the Frb variant. The most important mutation underlying this specificity is the Trp-to-Phe alteration at amino acid 2101 (data summarized in FIG. 14). The single mutation at this site to KTF accomodates the methallyl bump in rapamycin (EC50=9 nM) and markedly improves binding over KTW. Less pronounced contributions are made by the mutation at Thr 2098. Single mutation to KLW improves affinity about 5-fold over wild-type (22 nM), while the double mutant KLF (EC50=7.8 nM) has slightly improved binding to C20-MaRap over KTF. In the crystal structure of FKBP-rap in complex with the FRB domain, Trp2101 is partially buried between helix 4 and helix 1 of the FRB four helix bundle with the planar aromatic surface of the indole ring opposed to the coordinated triene between C17 and C22 of rapamycin. It appears then that the steric bulk of the methallyl group opposing the bulky indole ring be accomodated by substitution of the indole for the benzene ring of Phe. C20-MaRap breaks the triene and therefore permits greater torsional flexibility as well as adding bulk from the methallyl addition.

We tested other rapalogs derivatized at the interacting surface with FRB for specificity to FRB mutants at Trp2101 and Leu2098. Each of the rapalogs bumped at C16 have a measurable interaction with wild-type FRB; however, there is little or no binding between C16-BSrap and mutant FRBs with Phe substituted at amino acid 2101 (FIG. 14). There is an approximately four-fold improvement in EC50 for C16-BS rap for mutants with Leu substituted for Thr at amino acid 2098. A similar pattern of binding specificity was observed for C16-(S)iRap (AP21967) although this drug was significantly less effective than C16-BSrap against all mutants.

Like AP21967, C16-iRap has a bulky indole ring bumped at C16, but with the opposite stereochemistry. This molecule does not bind to FRB mutants with a Trp 2101 Phe substitution, but only in the context of Thr at 2098 (FIG. 14). Substitution of this residue with Leu fully rescues drug binding. There is only a minor, less than two fold, increase in EC50 by Thr 2098 to Leu substitution if Trp is at amino acid 2101. This indicates that the stereochemistry of the linkage of the bumps at C16 underlie the differences in specificity between C16-(R)iRap and C16-(S)iRap (and C16-BSrap as well). Bumps in either rotamer point into the interface with helix 4 of the FRB domain in close proximity to amino acids 2098 and 2101. While there is relatively little bulk added by substituting Phe for Trp, it appears that the Phe position is in close proximity to amino acid 2098 leaving little space to accomodate groups at C16 of rapamycin. Supporting this is the observation that the EC50 for rapamycin (with (S)methoxy at C16) is reduced in all mutants with the ‘TF’ combination. Close proximity between Phe at 2101 and Thr at 2098 are unlikely to clash per se as this combination of mutations does not influence the ΔΔG of the FRB domain. The binding of rapamycin and iRap is improved if Thr is changed to Leu probably because the long aliphatic side chain can point away from Phe providing enough space to accomodate groups at C16 of rapamycin especially in the R rotamer. Importantly, each of four variants at amino acid 2095 did not influence the binding specificity of rapalogs or materially affect (greater than 2 fold) the EC50 of a particular drug in combination with substitutions at 2098 or 2101.

Examples of stable FRB domains that bind C20-MaRap are KTF and ATF while PLF or TLF are most appropriate for inducible stabilization using C20-MaRap. Use of C16-iRap as the dimerizer, is most appropriate for the wild-type (KTW) maintaining a stable protein, while inducibile stabilization can be designed into the system with a PLF or TLF fusion. Systems with C16-BSrap can be destabilized partially with KLW fused to the target and the KTW will yield a stable fusion target.

The overlapping and in some cases unique specificity of rapalogs with differing bumps with given FRB mutants broadens the possibilities for the conditional regulation of protein function with rapalog directed dimerization. Multiple target proteins can be co-expressed as fusion different FRB variants to permit their coordinated, orthogonal regulation. As an example, two transcription factors can be expressed, one with KTW and the other with KTF with the exporter FKBP-NES coexpressed. In the presence of C16-BS-rap only the KTW fused target is exported while C20-MaRap will induce the export of the KTF fused target. Like most chemically regulated biologic systems the drug induced effect is reversed after treatment, this property can selectively utilized by treating with a drug that binds to both FRB mutants in a system, then switching to a drug that only interacts with one of the FRB fusions. In the presence of C16-iRap, both fusions are stabilized and active because this drug recruits FKBP to each mutant. When C16-iRap is washed out and replaced with C16-BSrap, FKBP-BSrap is no longer recruited to the KLF fusion to degrade while the KLW fusion remains stabilized. Similarly switching from C16-iRap to C20-MaRap selectively retains stabilized KLF fusions.

An alternative use for orthogonal induction of dimerization is one in which a target protein is fused to FKBP and regulated through the specific recruitment of one of multiple FRB tagged regulatory domains. GSK3β is a kinase and a key regulator of multiple intracellular signaling pathways normally localized both in the nucleus and cytoplasm. GSK3β was tagged with FKBP12 and coexpressed with two differing FRB mutants tagged with orthogonal subcellular targeting signals. TLW was fused to a 9 amino acid nuclear export sequence derived from the Rev protein of Human Immunodeficiency Virus to produce TLW-NES, and KTF was fused to the six amino acid nuclear localization sequence from SV40 Large T antigen (KTF-NLS). In the absence of drug, GSK3β was observed both in the cytoplasm and nucleus (FIG. 15). C20-Marap binds efficiently to KTF, but inefficiently to the TLW mutant. Incubation with 10 nM C20-Marap resulted in localization of GSK3β to the nucleus due to dimerization of the GSK3β-FKBP target with KTF-NLS in preference to TLW-NES. Stimulation with C16-BSRap had the opposite targeting reaction and GSK3β-FKBP was rapidly localized to the cytoplasm by selective recruitment of the TLW-NES. Such systems can be useful to parse nuclear functions from cytoplasmic functions for proteins such as GSK3β that have roles in multiple signaling pathways. Other examples in which a ‘one target, multiple effect’ system can be employed include transcriptional regulation in which the DNA binding domain of a transcription factor of interest is stripped of its domains that influence transcription and fused with FKBP. Selective recruitment of an activation domain or a repression domain (or both, in sequence) with specific rapalogs gives the experimentalist greater control of the timing of subsequent biochemical effects of stimulated or repressed transcription. Similarly, recruitment of kinases and phosphatases to nodes of signaling activity can facilitate subsequent proteomic analysis of the effect of this recruitment. The available technologies based on chemical induction of dimerization can be significantly broadened by the use of combinations of FRB fusions and rapalogs both for the characterization of biologic responses and biochemical effects.

Earlier studies (Liberles, S. D. et al. “Inducible Gene Expression and Protein Translocation Using Nontoxic Ligands Identified by a Mammalian Three-Hybrid Screen.” (1997) Published in Proc. Natl. Acad. Sci. USA 94, 7825-7830; Stankunas, K. et al. “Conditional Protein Alleles Using Knockin Mice and a Chemical Inducer of Dimerization.” (2003) Published in Mol. Cell 12, 1615-1624) found that drugs bumped at the C20 position of rapamycin could alleviate the cell-toxic effects of rapamycin and permit chemical induction of dimerization in vivo. The triple mutant selected to accomodate the bump on rapamycin (PLF) was found to destabilize the FRB domain and transfer this instability to proteins fused to the FRB domain (Stankunas, K. et al. “Conditional Protein Alleles Using Knockin Mice and a Chemical Inducer of Dimerization.” (2003) Published in Mol. Cell 12, 1615-1624), an effect reversed by rapalog recruitment of FKBP. The specificity of given mutant FRB domains for different rapalogs permits the combinatorial, orthogonal regulation of multiple FRB-fused target proteins. Several schemes are presented to take advantage of this improvement of dimerizer technology. Perhaps the most important advantage of this advancement to biologists is the coordinated, experimental regulation of multiple proteins in a biological pathway. Determining order in a pathway of interacting proteins such as a signal transduction cascade is perfomed genetically through tests of epistasis. In these tests, mutants in a pathway with different phenotypes are crossed and the mutant whose phenotype is evident is determined to be downstream in a pathway. These powerful experiments require that mutants, both loss or gain-of-function or at least with distinguishable phenotypes exist, a tall order in many mammalian systems. Chemical regulation of one FRB-tagged protein in combination or discord with another will broaden the ability of biologists to assign order in signal transduction pathways.

It is evident from the above results and descriptions that the subject invention provides a conditional allele system, where the amount of a functional protein can be controlled by varying the amount of a small stabilizing molecule that binds to a component of a fusion protein. The fusion protein is destabilized and degraded and is functional when stable and bound to the small stabilizing molecule. Knock-in small animals expressing the destabilized protein exhibit loss-of-function, while being able to develop through the fetal and neonate stages to early maturity, where the protein can be stabilized. Both GSK-3β^{FRB*} and Pax6^{FRB*} mice die at birth, phenocopying the respective knockout animals. This permits an evaluation of the role of the protein, permits drugs to be screened in a negative or positive background of the protein, and provides for observation of the changes resulting from the variation in amount of available protein as one varies the amount of small stabilizing molecule or antagonist to the small stabilizing molecule. In addition, the subject system permits investigation into the cellular pathways involving the destabilized protein, where changes in phenotype can be observed with variation in the presence or absence of the target protein. The system provides for the specific, rapid and reversible inactivation of target proteins and adds a major advance in the armamentarium of studying cellular pathways and evaluating the effect of drugs on such pathways.

More than one conditional allele (intends an allele of the same target protein or different proteins) may be introduced into cells and animals to evaluate the effect individually and in combination of the presence or absence of the alleles during development or other situations, where one is interested in the effect of a stimulus, e.g. candidate drug, on the development and differentiation of cells or hosts. The conditional alleles can be selected to be present or absent at different times during development to aid in the determination of how the target proteins affect the development of a host. In this way, pathways can be analyzed as to their contribution to the formation of organs and physical structure of a host.

All references referred to in the text are incorporated herein by reference as if fully set forth herein. All procedures disclosed in the references are incorporated as demonstrating the level of skill in the art to perform the procedures indicated in this application. The relevant portions associated with this document will be evident to those of skill in the art. Any discrepancies between this application and such reference will be resolved in favor of the view set forth in this application.

Although the invention has been described with reference to the above examples, it will be understood that modifications and variations are encompassed within the spirit and scope of the invention. Accordingly, the invention is limited only by the following claims.

1. A conditional allele system comprising a cell having at least one nucleic acid sequence expressing a fusion protein of a target protein and a destabilizing peptide, where the destabilizing peptide is characterized by: reducing the target protein-activity of the fusion protein; binding to a small stabilizing molecule that stabilizes the target protein-activity of the fusion protein; wherein said target protein functions when bound to said small stabilizing molecule; and a small non-toxic stabilizing molecule for each fusion protein that when bound to said destabilizing peptide stabilizes the target protein-activity of said fusion protein for said fusion protein to function.

2. A conditional allele system according to claim 1, wherein said reducing the target protein-activity results from the degradation of said fusion protein.

3. A conditional allele system according to claim 1, wherein said destabilizing peptide is a mutated version of the FRB domain (of mTor).

4. A conditional allele system according to claim 3, wherein said small stabilizing molecule is rapamycin or a rapamycin analogue comprising a leucine substitution at position 2098..

5. A conditional allele system according to claim 1, wherein the wild-type target protein is not expressed in said cell.

6. A conditional allele system according to claim 1, wherein said cell is mammalian, said small stabilizing molecule recruits an endogenous protein for said stabilizing; and wherein the combination of said fusion protein, small stabilizing molecule and endogenous protein is functional as said target protein.

7. A conditional allele system according to claim 1, having two of said nucleic acid sequences expressing two fusion proteins comprising different target proteins and different destabilizing peptides and there being two small stabilizing molecules, at least one substantially non-cross-reacting, binding to the two different destabilizing peptides.

8. A conditional allele system comprising a nucleic acid encoding a fusion protein that comprises a target protein and a peptide that destabilizes the fusion protein when present in a cell; and a small non-toxic stabilizing molecule capable of entering said cell and binding to said destabilizing peptide of said fusion protein and stabilizing said fusion protein resulting in a functioning target protein.

9. A conditional allele system according to claim 7, wherein said destabilizing peptide is a mutated version of the FRB domain (of mTor).

10. A mammalian host comprising a cell according to claim 1, wherein said cell is mammalian.

11. A mammalian host according to claim 10, wherein said host is murine.

12. A mammalian host according to claim 9, wherein said destabilizing peptide is a mutated version of the FRB domain (of mTor).

13. A mammalian host according to claim 10, wherein said target gene is GSK-3β or Pax6.

14. A fusion protein comprising a fusion of a target protein and a destabilizing peptide to form said fusion protein, where the destabilizing peptide is characterized by: causing the degradation of the fusion protein when present in a cell; binding to a small molecule that stabilizes the fusion protein; and said target protein functions when bound to said small stabilizing molecule.

15. A method of screening for the effect of a drug on a target protein function using a cell comprising a fusion protein, where said fusion protein is the fusion of at least an active portion of said target protein having said protein function and a destabilizing peptide, where the destabilizing peptide is characterized by: diminishing said protein function of said fusion protein; and binding to a small non-toxic molecule capable of entering said cell, that results in the enhancement of said protein function; said method comprising: adding said drug to said cell in the presence of said small non-toxic molecule and determining at least one phenotypic characteristic as compared to said phenotypic property in the absence of said small non-toxic molecule, whereby the effect of said drug in the presence and absence of said protein function is determined.

16. A method according to claim 15, wherein said small non-toxic molecule recruits an endogenous protein for stabilizing said fusion protein.

17. A method for determining the effect of the presence of a protein function of a target protein in a cell using a fusion protein, said fusion protein comprising the fusion of at least an active portion of said target protein having said protein function and a destabilizing peptide, where the destabilizing peptide is characterized by; binding to a small non-toxic molecule that results in the enhancement of said protein function of said fusion protein, said method comprising: adding said small non-toxic molecule to said cell and measuring at least one characteristic of said cell as compared to the absence of said small non-toxic molecule.

18. A method for determining a biological function of at least one target protein in a mammalian host where said target protein is replaced with a fusion protein according to claim 14, said fusion protein comprising a target protein and a destabilizing peptide, said destabilizing peptide binding to and stabilized by a small non-toxic molecule, said method comprising: growing said host in a supportive environment for growth, while either not administering or administering said small non-toxic molecule in a predetermined amount into said environment, whereby the functional level of said fusion protein is modulated ; and determining the effects on the biology of said host of the presence, absence or amount of said small non-toxic molecule.

19. A method according to claim 18, there being two target proteins each stabilized by a different small non-toxic molecule, and wherein said functional level of each of said fusion proteins is modulated by varying the amount of each of said small non-toxic molecules.

20. A method according to claim 19, wherein said host is embryonic or fetal.

21. A method according to claim 18, wherein said environment is a murine uterus.

22. A mammalian cell comprising a genetic construct expressing an unstable protein involved in the development of a fetus stabilized by a small non-toxic organic molecule of less than 5 kDa.
